(19) United States
(12) Patent Application Publication
(10) Pub. No.: US 2025/0186727 A1
(43) Pub. Date: Jun. 12, 2025

(54) POSITIONING AND STABILISING STRUCTURES FOR PATIENT INTERFACES

(71) Applicant: RESMED ASIA PTE. LTD., Singapore (SG)

(72) Inventors: Michiel KOOIJ, Sydney (AU); Thontira SUPAOPASPHUN, Singapore (SG); Mohankumar Krishnan VALIYAMBATH, Singapore (SG); Bangzheng TAN, Singapore (SG)

(21) Appl. No.: 18/686,042

(22) PCT Filed: Aug. 25, 2022
(86) PCT No.: PCT/AU2022/050997

§ 371 Date: Feb. 23, 2024

(30) Foreign Application Priority Data
Aug. 25, 2021 (AU) 2021902713

(51) Int. Cl. A61M 16/06; A61M 16/08

(52) U.S. Cl. CPC A61M 16/0622; A61M 16/0683; A61M 16/0875

(57) ABSTRACT

The present technology relates to a positioning and stabilising structure for a patient interface, the positioning and stabilising structure comprising a conduit configured to permit delivery of pressurised air to the interior of the plenum chamber and a pad attached to the conduit. Other aspects of the present technology relate to a patient interface comprising a plenum chamber, a seal-forming structure configured to form a seal with a region of the patient's face surrounding an entrance to the patient's airways, and the positioning and stabilising structure comprising the conduit and the pad attached to the conduit.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, their use, and methods of manufacturing the devices and apparatus or components therefor.

### 1.2 DESCRIPTION OF THE RELATED ART

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, noisy or difficult to use a patient may not comply with therapy.

Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 1.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as an “elephant trunk” style of interface.

Some patients find such interfaces to be unsightly and are consequently deterred from wearing them, reducing patient compliance. Additionally, conduits connecting to an interface at the front of a patient's face may sometimes be vulnerable to becoming tangled up in bed clothes.

1.2.3.1.4 Pressurised Air Conduit used for Positioning/Stabilising the Seal-Forming Structure

An alternative type of treatment system which seeks to address these problems comprises a patient interface in which a tube that delivers pressurised air to the patient's airways also functions as part of the structure to position and stabilise the seal-forming portion of the patient interface to the appropriate part of the patient's face, also referred to as “headgear”. This type of patient interface may be referred to as incorporating a “headgear conduit system”. References to “headgear conduit system” are to be understood to refer to components which may otherwise be known as “headgear tubing”, “headgear tubes”, or “conduit headgear”, amongst others.

The headgear conduit system may comprise at least one conduit, which may be referred to as a ‘headgear conduit’ or ‘conduit’. Preferably, the headgear conduit system comprises a pair of headgear conduits. Such patient interfaces allow the conduit in the air circuit providing the flow of pressurised air from a respiratory pressure therapy device to connect to the patient interface in a position other than in front of the patient's face. One example of such a treatment system is disclosed in US Patent Publication No. 2007/0246043, the contents of which are incorporated herein by reference, in which the conduit connects to the headgear conduit(s) in the patient interface through a connection port positioned in use on top of the patient's head.

Patient interfaces incorporating a headgear conduit system may provide some advantages, for example avoiding a conduit connecting to the patient interface at the front of a patient's face, which may be unsightly and obtrusive. However, it is desirable for patient interfaces incorporating a headgear conduit system to be comfortable for a patient to wear over a prolonged duration when the patient is asleep while forming an effective seal with the patient's face.

Existing headgear conduit systems may have textile sleeves formed of a relatively soft material which are slid over or positioned to wrap around the conduit. The softer sleeve provides a patient contacting surface for comfort when donning the headgear conduit. However, making sleeves requires larger amounts of textile material to be used. They may also be moved out of place, crease or fold or not be repositioned properly by a user after removal. This may cause discomfort and reduce compliance by a user. Furthermore, the sleeves are relatively thin and to increase the cushioning or padding effect provided by the sleeve requires thickening the sleeve by providing more cushioning or padding layers to it. This may make the headgear conduit bulky and obtrusive, and uncomfortable to wear.

In yet other examples of existing headgear conduit systems, one or more pads may be attached to the headgear conduit, for example the pad(s) may be moulded, over-moulded, co-moulded or attached using adhesives to a conduit. References to “pad” are to be understood to refer to components which may otherwise be known as “padding”, “cushion” or “cushioning”, amongst others. These are components that provide a cushioning or padding effect. However, the attachment of the pad(s) to conduits made of certain materials can be complicated and difficult to achieve. This may increase manufacturing time and costs. There may also be limitations to the type of materials that can be bonded together-which limits the types of pads that can be provided for conduits.

Furthermore, existing headgear conduit systems may have edges, seams or uneven joints on the conduits, pads, and the junction between two components. These edges, seams or joints can be rough, sharp, uneven and/or obtrusive. These rough, sharp, uneven edges, seams or joints may be created when cutting the pad to a desired size and shape before attaching the pad to the conduit. These sharp, uneven edges, seams or joints are undesirable and may irritate and mark the patient's skin during use, causing discomfort to the patient, and which may as a result reduce compliance with therapy. In addition, these edges, seams or joints may also be unsightly and reduce the aesthetics of the product which could further reduce compliance.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:2007,
10 cmH2O pressure at 1 m)

| | | A-weighted | A-weighted | |
|---|---|---|---|---|
| | | sound power | sound pressure | |
| | Mask | level dB(A) | dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage ™ (*) | | | | |
| ResMed | nasal | 36 (3) | 28 (3) | 2000 |
| UltraMirage ™ | | | | |
| ResMed | nasal | 32 (3) | 24 (3) | 2002 |
| Mirage | | | | |
| Activa ™ | | | | |
| ResMed | nasal | 30 (3) | 22 (3) | 2008 |
| Mirage | | | | |
| Micro ™ | | | | |
| ResMed | nasal | 29 (3) | 22 (3) | 2008 |
| Mirage ™ | | | | |
| SoftGel | | | | |
| ResMed | nasal | 26 (3) | 18 (3) | 2010 |
| Mirage ™ FX | | | | |
| ResMed | nasal | 37 | 29 | 2004 |
| Mirage Swift ™ | pillows | | | |
| (*) | | | | |
| ResMed | nasal | 28 (3) | 20 (3) | 2005 |
| Mirage Swift ™ | pillows | | | |
| II | | | | |
| ResMed | nasal | 25 (3) | 17 (3) | 2008 |
| Mirage Swift ™ | pillows | | | |
| LT | | | | |
| ResMed AirFit | nasal | 21 (3) | 13 (3) | 2014 |
| P10 | pillows | | | |

(* one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

Another aspect of certain forms of the present technology is to provide methods and/or apparatus that improve the compliance of patients with respiratory therapy.

Another aspect of certain forms of the present technology is to provide methods and/or apparatus that improve the comfort of patients during respiratory therapy.

Another aspect of the present technology relates to a positioning and stabilising structure configured to hold a seal-forming structure of a patient interface in a sealing position on a patient's face. The positioning and stabilising structure may comprise a conduit. The positioning and stabilising structure may further comprise a pad.

In examples:
The conduit may be configured to permit delivery of a flow of air at a therapeutic pressure into a plenum chamber of the patient interface through an inlet formed in the plenum chamber.
The conduit may comprise first and second conduits.
The positioning and stabilising structure may be configured such that, in use, the first conduit may be positioned on one side of the patient's face and the second conduit may be positioned on the other side of the patient's face.
The positioning and stabilising structure may further comprise at least one headgear strap attached to the conduit.

In examples:
The conduit may comprise a patient facing side and a non-patient facing side.
An outer surface of the conduit may comprise a first outer surface region and an adjacent second outer surface region.
The outer surface of the conduit may be configured such that, when the pad is positioned on the first outer surface region, the outer surface of the pad and an outer surface of the second outer surface region are substantially flush.
The outer surface of the conduit may be configured such that the first outer surface region is recessed from the second outer surface region.
The conduit may comprise a connecting wall which connects the first outer surface region and the second outer surface region.
The conduit may comprise first and second connecting walls connecting lateral edges of the first outer surface region with respective lateral edges of the second outer surface region.

In examples:
The pad may comprise a conduit facing surface, an opposing patient facing surface, opposing lateral edges and opposing end edges.
The conduit facing surface of the pad may be attached to an outer surface of the conduit on the patient facing side.
The patient facing surface of the pad may contact the patient's head and/or face in use.

In examples:
The first outer surface region may be located on the patient facing side and the second outer surface region may be located on the non-patient facing side.
The pad may be positioned on the first outer surface region of the conduit between the first and second connecting walls.

In examples:
At least one of the edges of the pad may be folded under itself to create a folded region.
The pad may comprise a first pad region and a second pad region.
The first pad region may be folded under the second pad region such that at least one of the edges of the pad is positioned between the outer surface of the conduit and a conduit facing surface of the second pad region, and such that a patient facing surface of the second pad region contacts the patient's head and/or face in use.
The first pad region may provide the folded region.
The outer surface of the conduit may be configured such that, when the pad is positioned on the first outer surface region, a joint region between the patient facing surface of the second pad region and an outer surface of the second outer surface region is substantially flush.
The conduit may comprise a groove.
A groove may be formed in the outer surface of the conduit.
The groove may be formed in the first outer surface region.
The groove may extend along at least a portion of a length of the conduit.
The first pad region/folded region may be positioned in the groove.

In examples:
The conduit may comprise a notch.
An outer surface of the conduit may be configured such that a first outer surface of the conduit overhangs a second outer surface of the conduit and creates a notch extending between the first outer surface and the second outer surface.
The notch may extend along at least a portion of a length of the conduit.
At least one of the edges of the pad may be positioned in the notch.

In examples:
The pad may be positioned on the conduit such that at least one of the edges of the pad is positioned, in use, to face in a direction extending away from the patient's face and/or head.
At least a portion of the first outer surface region is substantially flat.
At least a portion of the first outer surface region is substantially convex-shaped.
At least a portion of the second outer surface region is substantially convex-shaped.
The connecting wall may be convex-shaped.
The connecting wall and the first outer surface region may be shaped such that a joint region between the connecting wall and the first outer surface region is concave-shaped.
The joint region may be on the patient facing side of the conduit.
At least one of the edges of the pad may be positioned on the joint region.
A first portion of the first outer surface region may be convex-shaped and an adjacent second portion of the first outer surface region may be concave-shaped.
The second portion may be located between the second outer surface region and the first portion.
At least one of the edges of the pad may be positioned on the second portion.

In examples:
The first outer surface region may be located on the non-patient facing side and the second outer surface region may be located on the patient facing side.
The pad may be positioned to overlie the second outer surface region such that at least one of the edges of the pad is positioned on the first outer surface region.
The pad may be positioned to overlie the second outer surface region and the first and second connecting walls.
The pad may be positioned to overlie the second outer surface region such that at least one of the edges of the pad is positioned on the connecting wall.

In examples:
The conduit may be formed from silicone.
The positioning and stabilising structure may comprise a layer of silicone material.
The layer of silicone material may be attached to an outer surface of the conduit on the patient facing side.
The conduit facing surface of the pad is attached to the layer of silicone material.
The conduit facing surface of the pad is attached to the layer of silicone material such that the layer of silicone material is positioned between the outer surface of the conduit on the patient facing side and the conduit facing surface of the pad.
The layer of silicone material may be applied to the outer surface of the conduit in a partially cured form.
The pad may be positioned on the partially cured layer of silicone material such that, when the partially cured layer of silicone material cures, the conduit, the layer of silicone material, and the pad are attached together.
The layer of silicone material may be removably attached to the outer surface of the conduit.

Another aspect of the present technology relates to a positioning and stabilising structure configured to hold a seal-forming structure of a patient interface in a sealing position on a patient's face. The positioning and stabilising structure may comprise a headgear member. The positioning and stabilising structure may further comprise a pad.

In examples:
The headgear member may be configured to extend, in use, over a portion of the patient's face and/or head.
The headgear member may comprise a patient facing side and a non-patient facing side.

In examples:
The pad may comprise a headgear facing surface, an opposing patient facing surface, opposing lateral edges and opposing end edges.
The pad may be removably attached to an outer surface of the patient facing side of the headgear member.

In examples:
The headgear member may comprise the conduit according to any one or more of the previously described aspects and/or examples of the invention.
The headgear member may comprise a headgear strap or tie.
The outer surface of the headgear member may comprise a first outer surface region and an adjacent second outer surface region.
The headgear member may comprise first and second connecting walls connecting lateral edges of the first outer surface region with respective lateral edges of the second outer surface region.
The pad may be positioned on the first outer surface region between the first and second connecting walls.
The first outer surface region may be located on the patient facing side of the headgear member.
The second outer surface region may be located on the non-patient facing side.
At least a portion of the first outer surface region may be substantially flat.
At least a portion of the first outer surface region may be substantially convex-shaped.
A joint region between the first outer surface region and the second outer surface region may be substantially convex-shaped.
The first and second connecting walls may be substantially convex-shaped.

In examples:
One of the pad and the headgear member may comprise a projecting member formed on the headgear facing surface of the pad or the outer surface of the patient facing side of the headgear member, and the other one of the pad and the headgear member may comprise an aperture formed in the headgear facing surface of the pad or the outer surface of the patient facing side of the headgear member.
The aperture may be configured to receive at least a portion of the projecting member.
The projecting member may be configured to engage with a surface of the pad or a surface of the headgear member which at least partially defines the aperture.
The pad may comprise a padding portion and a connector portion.
The connector portion may be formed separately from and attached to the padding portion.
The connector portion may comprise the projecting member or the aperture.
The projecting member may be a first projecting member and the pad or headgear member may comprise a second projecting member.
The aperture may be a first aperture and the pad or headgear member may comprise a second aperture.
The first aperture may be configured to receive at least a portion of the first projecting member.
The second aperture may be configured to receive at least a portion of the second projecting member.
The first projecting member may be spaced apart from the second projecting member along a perimeter of the patient facing surface of the headgear member or a width of the headgear facing surface of the pad.
The first aperture may be spaced apart from the second aperture along the perimeter of the patient facing surface of the headgear member or the width of the headgear facing surface of the pad.
The aperture may comprise a groove extending along at least a portion of a length of the headgear facing surface of the pad or a length of the patient facing surface of the headgear member.
The projecting member may extend along at least a portion of a length of the headgear facing surface of the pad or a length of the patient facing surface of the headgear member.
The pad may comprise the projecting member and the aperture may be formed in the outer surface of the headgear member.

Another aspect of the present technology relates to a positioning and stabilising structure configured to hold a seal-forming structure of a patient interface in a sealing position on a patient's face. The positioning and stabilising structure may comprise a conduit. The conduit may be configured to permit delivery of a flow of air at a therapeutic pressure into a plenum chamber of the patient interface through an inlet formed in the plenum chamber. The conduit may comprise a patient facing side and a non-patient facing side. The conduit may be formed from silicone. The positioning and stabilising structure may further comprise a layer of silicone material attached to an outer surface of the conduit on the patient facing side. The positioning and stabilising structure may further comprise a pad. The pad may comprise a conduit facing surface, an opposing patient facing surface, opposing lateral edges and opposing end edges. The conduit facing surface of the pad may be attached to the layer of silicone material such that the layer of silicone material is positioned between the outer surface of the conduit on the patient facing side and the conduit facing surface of the pad.

Another aspect of the present technology relates to a positioning and stabilising structure configured to hold a seal-forming structure of a patient interface in a scaling position on a patient's face. The positioning and stabilising structure may comprise a conduit. The conduit may be configured to permit delivery of a flow of air at a therapeutic pressure into a plenum chamber of the patient interface through an inlet formed in the plenum chamber. The conduit may comprise a patient facing side and a non-patient facing side. The positioning and stabilising structure may further comprise a pad. The pad may comprise a conduit facing surface, an opposing patient facing surface, opposing lateral edges and opposing end edges. The conduit facing surface of the pad may be attached to an outer surface of the conduit on the patient facing side. The pad may comprise a first pad region and a second pad region. The first pad region may be folded under the second pad region such that at least one of the edges of the pad is positioned between the outer surface of the conduit and a conduit facing surface of the second pad region, and such that a patient facing surface of the second pad region contacts the patient's head and/or face in use.

Another aspect of the present technology relates to a positioning and stabilising structure configured to hold a seal-forming structure of a patient interface in a sealing position on a patient's face. The positioning and stabilising structure may comprise a conduit. The conduit may be configured to permit delivery of a flow of air at a therapeutic pressure into a plenum chamber of the patient interface through an inlet formed in the plenum chamber. The conduit may comprise a patient facing side and a non-patient facing side. The positioning and stabilising structure may further comprise a pad. The pad may comprise a conduit facing surface, an opposing patient facing surface, opposing lateral edges and opposing end edges. The conduit facing surface of the pad may be attached to an outer surface of the conduit on the patient facing side. The patient facing surface of the pad may contact the patient's head and/or face in use. The outer surface of the conduit may be configured such that a first outer surface of the conduit overhangs a second outer surface of the conduit and creates a notch extending between the first outer surface and the second outer surface. At least one of the edges of the pad may be positioned in the notch.

Another aspect of the present technology relates to a positioning and stabilising structure configured to hold a seal-forming structure of a patient interface in a sealing position on a patient's face. The positioning and stabilising structure may comprise a conduit. The conduit may be configured to permit delivery of a flow of air at a therapeutic pressure into a plenum chamber of the patient interface through an inlet formed in the plenum chamber. The conduit may comprise a patient facing side and a non-patient facing side. The positioning and stabilising structure may further comprise a pad. The pad may comprise a conduit facing surface, an opposing patient facing surface, opposing lateral edges and opposing end edges. The conduit facing surface of the pad may be attached to an outer surface of the conduit on the patient facing side such that the patient facing surface of the pad contacts the patient's head and/or face in use. The outer surface of the conduit may comprise a first outer surface region and a second outer surface region. The outer surface of the conduit may be configured such that the first outer surface region is recessed from the second outer surface region. The pad may be positioned to lie over the second outer surface region. The outer surface of the conduit may further be configured such that at least one of the edges of the pad is positioned, in use, to face in a direction extending away from the patient's face and/or head.

Another aspect of the present technology relates to a positioning and stabilising structure configured to hold a seal-forming structure of a patient interface in a sealing position on a patient's face. The positioning and stabilising structure may comprise a conduit. The conduit may be configured to permit delivery of a flow of air at a therapeutic pressure into a plenum chamber of the patient interface through an inlet formed in the plenum chamber. The conduit may comprise a patient facing side and a non-patient facing side. The positioning and stabilising structure may further comprise a pad. The pad may comprise a conduit facing surface, an opposing patient facing surface, opposing lateral edges and opposing end edges. The patient facing surface of the pad may be attached to an outer surface of the conduit on the patient facing side such that the patient facing surface of the pad contacts the patient's head and/or face in use. The outer surface of the conduit may comprise a first outer surface region, a second outer surface region, and a connecting wall which connects the first outer surface region and the second outer surface region. The outer surface of the conduit may be configured such that the first outer surface region is recessed from the second outer surface region. The pad may be positioned on the first outer surface region. The connecting wall and the second outer surface region may be shaped such that a joint region between the connecting wall and the second outer surface region is concave-shaped.

Another aspect of the present technology relates to a positioning and stabilising structure configured to hold a seal-forming structure of a patient interface in a scaling position on a patient's face. The positioning and stabilising structure may comprise a headgear member. The headgear member may be configured to extend, in use, over a portion of the patient's face and/or head. The headgear member may comprise a patient facing side and a non-patient facing side. The positioning and stabilising structure may further comprise a pad. The pad may comprise a headgear facing surface, an opposing patient facing surface, opposing lateral edges and opposing end edges. The pad may be removably attached to an outer surface of the patient facing side of the headgear member. One of the pad and the headgear member may comprise a projecting member formed on the headgear facing surface of the pad or the outer surface of the patient facing side of the headgear member, and the other one of the pad and the headgear member may comprise an aperture formed in the headgear facing surface of the pad or the outer surface of the patient facing side of the headgear member. The aperture may be configured to receive at least a portion of the projecting member. The projecting member may be configured to engage with a surface of the pad or a surface of the headgear member which at least partially defines the aperture.

Another aspect of the present technology relates to a patient interface. The patient interface may comprise a plenum chamber. The plenum chamber may be pressurisable to a therapeutic pressure of at least 6 cmH₂O above ambient air pressure. The plenum chamber may comprise an inlet. The inlet may be configured to receive a flow of air at the therapeutic pressure for breathing by a patient. The patient interface may further comprise a seal-forming structure. The seal-forming structure may be configured to form a seal with a region of the patient's face surrounding an entrance to the patient's airways. The seal-forming structure may be further configured to maintain said therapeutic pressure in the plenum chamber throughout the patient's respiratory cycle in use. The patient interface may further comprise a positioning and stabilising structure. The positioning and stabilising structure may comprise the positioning and stabilising structure according to any one or more of the previously described aspects and/or examples of the invention.

One form of the present technology relates to a patient interface system, wherein the system comprises:
a plenum chamber;
a seal-forming structure; and
a headgear conduit system comprising:
   a conduit formed at least in part from silicone,
   a layer of silicone material attached to the part of the conduit formed from silicone, and
wherein a pad is attached to the layer of silicone material.

Another form of the present technology relates to a headgear conduit system for a positioning and stabilising structure for a patient interface system, wherein the headgear conduit system comprises:
a conduit which provides a surface formed from silicone, and
a layer of silicone material attached to the surface formed from silicone,
wherein a pad is attached to the layer of silicone material.

Another form of the present technology relates to a patient interface system, wherein the system comprises:
a plenum chamber;
a seal-forming structure; and
a headgear conduit system comprising:
   a conduit, and
   a pad attached to the conduit,
      wherein an edge region of the pad is folded and tucked under itself to create a substantially smooth edge.

Another form of the present technology relates to a headgear conduit system comprising:
a conduit, and
a pad attached to the conduit,
wherein an edge region of the pad is folded and tucked under itself to create a substantially smooth edge.

Another form of the present technology relates to a patient interface system, wherein the system comprises:
a plenum chamber;
a seal-forming structure; and
a headgear conduit system comprising:
   a conduit, and
   a pad attached to the conduit,
      wherein the conduit comprises a cavity, and
      further wherein an edge region of the pad is positioned in the cavity.

Another form of the present technology relates to a headgear conduit system comprising:
a conduit, and
a pad attached to the conduit,
   wherein the conduit comprises a cavity, and
   further wherein an edge region of the pad is positioned in the cavity.

Another form of the present technology relates to a patient interface system, wherein the system comprises:
a plenum chamber;
a seal-forming structure; and
a headgear conduit system comprising:
   a conduit, and
   a pad attached to the conduit,
      wherein the conduit comprises a recess in which the pad is at least partially positioned, and wherein an edge region of the pad is folded and tucked under itself to create a smooth joint between an edge of the recess and the pad.

Another form of the present technology relates to a headgear conduit system comprising:
a conduit, and
a pad attached to the conduit,
   wherein the conduit comprises a recess in which the pad is at least partially positioned, and wherein an edge region of the pad is folded and tucked under itself to create a smooth joint between an edge of the recess and the pad.

Another form of the present technology relates to a patient interface system, wherein the system comprises:
a plenum chamber;
a seal-forming structure; and
a headgear conduit system comprising:
   a conduit, and
   a pad attached to the conduit,
      wherein the conduit comprises a recess in which the pad is at least partially positioned, and wherein the recess comprises an overhang, and a gap between the overhang and a wall defining the recess defines a cavity, and
      further wherein an edge region of the pad is positioned in the cavity.

Another form of the present technology relates to a headgear conduit system comprising:
a conduit, and
a pad attached to the conduit,
   wherein the conduit comprises a recess in which the pad is at least partially positioned, and wherein the recess comprises an overhang, and an edge region of the pad is positioned under the overhang.

Another form of the present technology relates to a patient interface system, the system comprises:
a plenum chamber;
a seal-forming structure; and
a headgear conduit system comprising:
   a conduit, and
   a pad attached to the conduit,
      wherein the conduit comprises an edge, and wherein the pad is positioned to lie over the top of the edge.

Another form of the present technology relates to a headgear conduit system comprising:
a conduit, and
a pad attached to the conduit,
   wherein the conduit comprises an edge, and wherein the pad is positioned to lie over the top of the edge.

Another form of the present technology comprises a positioning and stabilising structure for a patient interface system, wherein the positioning and stabilising structure comprises the headgear conduit system described herein.

In an example, the positioning and stabilising structure may further comprise at least one headgear strap.

In an example, the plenum chamber may be pressurisable to a therapeutic pressure of at least 6 cmH₂ O above ambient air pressure.

In an example, the plenum chamber may comprise an inlet configured to receive a flow of air at the therapeutic pressure for breathing by a patient.

In an example, the seal-forming structure may be configured to form a seal with a region of the patient's face surrounding an entrance to the patient's airways. For instance, the seal-forming structure may be configured to form a seal with or around the patient's nose and/or mouth.

In an example, the seal-forming structure may be configured to maintain a therapeutic pressure in the plenum chamber throughout the patient's respiratory cycle in use.

In an example, the headgear conduit system may be configured to permit delivery of pressurised air to the inlet.

In an example, the headgear conduit system may be configured to position and stabilise the seal-forming structure on the patient's face.

In an example, the conduit may be formed at least in part from silicone. For instance, the conduit provides a surface formed from silicone.

In an example, a layer of silicone material may be positioned between the conduit and the pad.

In an example, a layer of silicone material may be attached to the part of the conduit formed from silicone.

In an example, the pad may be attached to the layer of silicone material.

In an example, the pad may comprise a layer of silicone material which is attached to the part of the conduit formed from silicone.

In an example, the pad may comprise at least a layer of cushion material.

In an example, the headgear conduit system may be configured to limit or prevent formation of sharp edges.

In an example, the pad and/or the conduit may be configured and arranged to limit or prevent formation of sharp edges at the junction between an edge of the pad and a surface of the conduit.

In an example, the pad may sit substantially flush with a portion of the conduit.

In an example, the conduit may comprise a recess.

In an example, the pad may be at least partially positioned in the recess.

In an example, the pad may be seated in the recess. For instance, the recess may be configured so that the pad may be seated in the recess and no portion of the pad is folded, tucked under itself, or positioned under a portion of the conduit when the pad is positioned in the recess.

In an example, the pad and/or conduit may be configured and arranged to provide a substantially smooth edge.

In an example, an edge region of the pad may be folded and tucked under itself to create a substantially smooth edge.

In an example, an edge region of the pad may be folded and tucked under itself to create a joint between an edge of the recess and the pad. For instance, the recess may be configured so that the edge region of the pad is folded and tucked under itself to create a folded region. The folded region may have a substantially smooth edge. The substantially smooth edge may be positioned to abut the recess.

In an example, the conduit may comprise a cavity.

In an example, an edge region of the pad may be positioned in the cavity.

In an example, the conduit comprises an overhang. For instance, a gap between the overhang and another portion of the conduit may define the cavity.

In an example, the recess may comprise the overhang. For instance, a gap between the overhang and a wall defining the recess may define the cavity.

In an example, an edge region of the pad may be positioned under the overhang.

In an example, a joint between an edge region of the pad and the conduit may be positioned, in use, away from the patient's face and/or head.

In an example, the conduit may comprise an edge. For instance, the pad may be positioned to lie over the top of the edge.

In an example, the edge may be provided by a surface of the conduit defining a/the recess.

Another aspect of one form of the present technology is a patient interface that is moulded or otherwise constructed with a perimeter shape which is complementary to that of an intended wearer.

An aspect of one form of the present technology is a method of manufacturing apparatus.

Another form of the present technology relates to a method of manufacturing a headgear conduit system, the method comprising the following steps, occurring in any order:
(a) forming a pad;
(b) forming at least a portion of a conduit from a silicone material; and
(c) attaching the pad to the conduit, wherein the step of attaching the pad to the conduit attaches a layer of silicone material to the conduit.

In examples, step (a) may comprise forming a pad comprising at least a layer of cushion material.

In examples, step (b) may comprise one or more of the steps of:
forming the conduit at least in part from silicone;
forming a conduit which provides at least a surface formed from silicone;
forming a conduit which comprises a recess;
forming a recess comprising an overhang;
forming a conduit with an edge;
forming a conduit with a protruding portion to provide the conduit edge.

In examples, step (c) may comprise one or more of the steps of:
attaching the pad to the silicone portion of the conduit;
attaching the pad to the conduit surface formed from silicone;
attaching a layer of silicone material to the silicone portion of the conduit and the pad;
attaching a layer of silicone material to the pad and then attaching the pad to the silicone portion of the conduit;
positioning the pad in the recess to limit or prevent formation of sharp edges at the junction between an edge of the pad and a surface of the conduit;
folding an edge region of the pad and tucking it under itself to create a folded region which has a substantially smooth edge;
positioning the substantially smooth edge to abut the recess;
positioning an edge region of the pad under the overhang;
positioning a joint between an edge region of the pad and the conduit away from the patient's face and/or head in use;
positioning the pad to lie over the top of the conduit edge.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

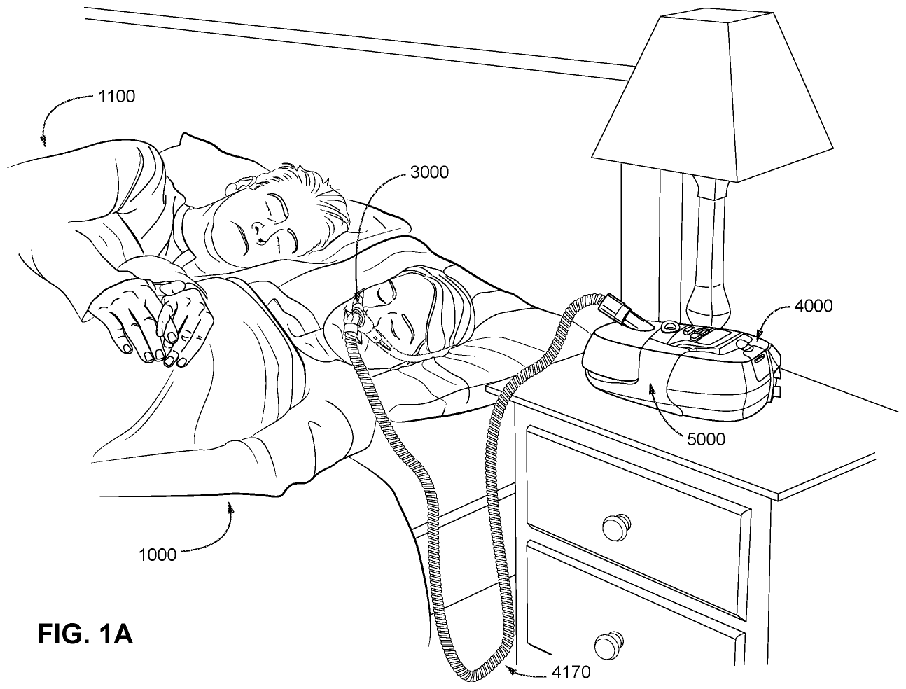

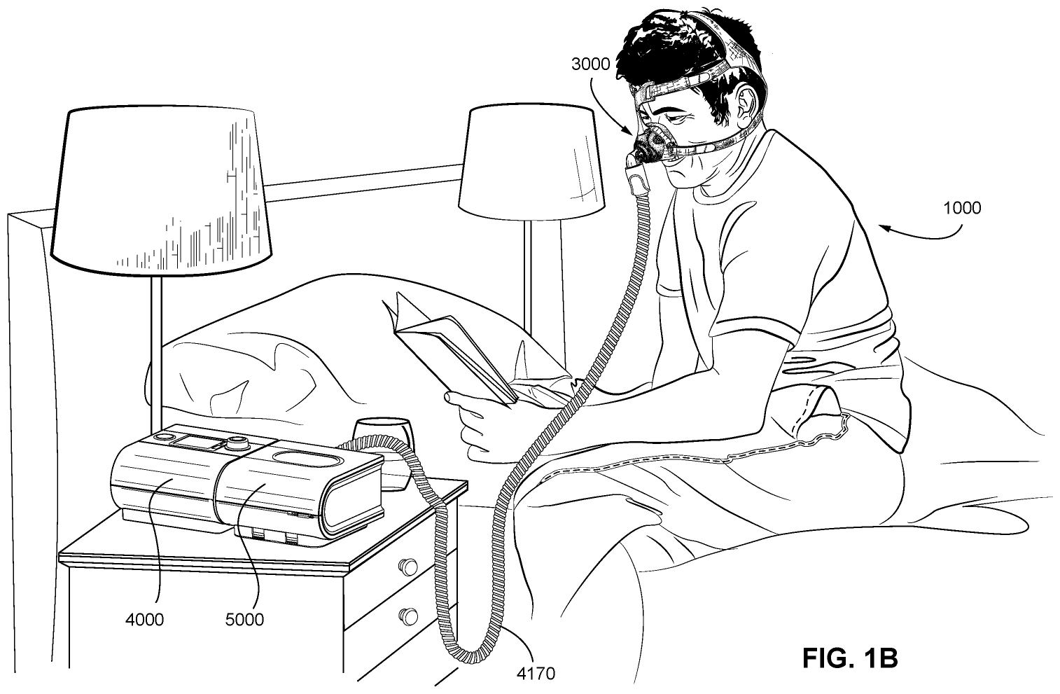

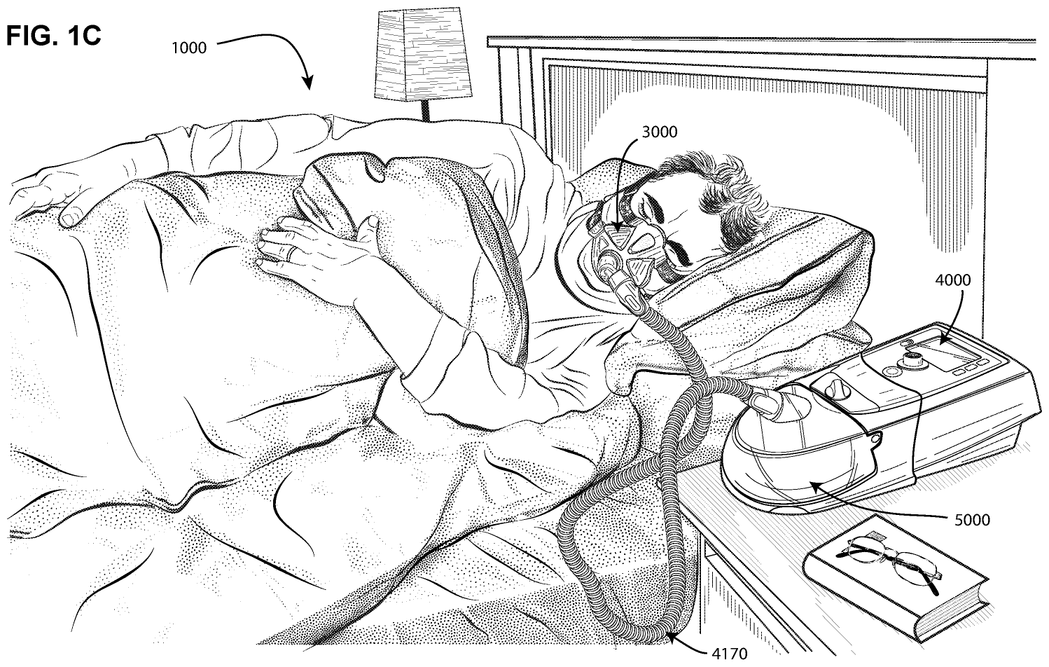

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 RESPIRATORY SYSTEM AND FACIAL ANATOMY

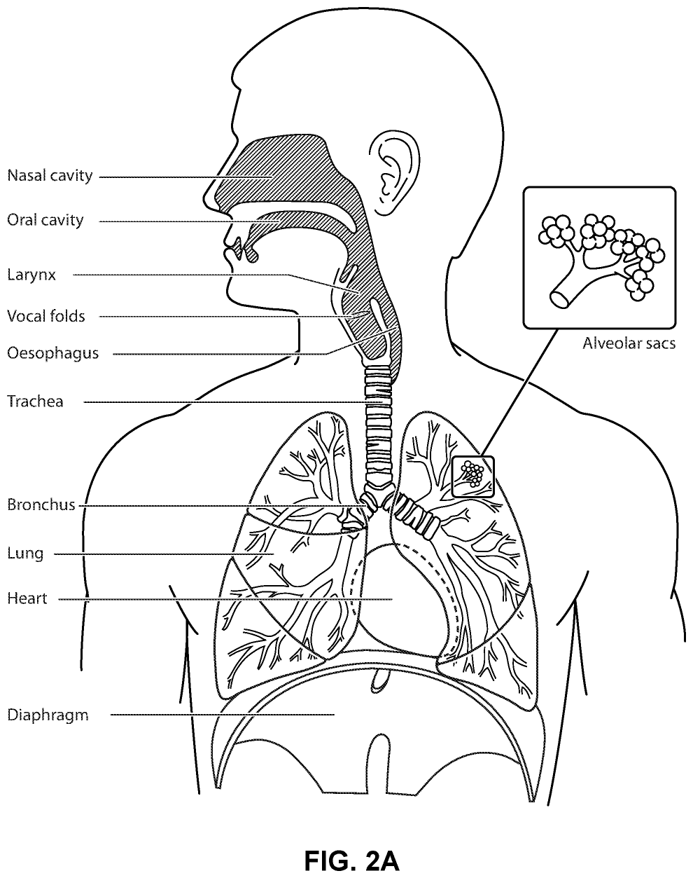

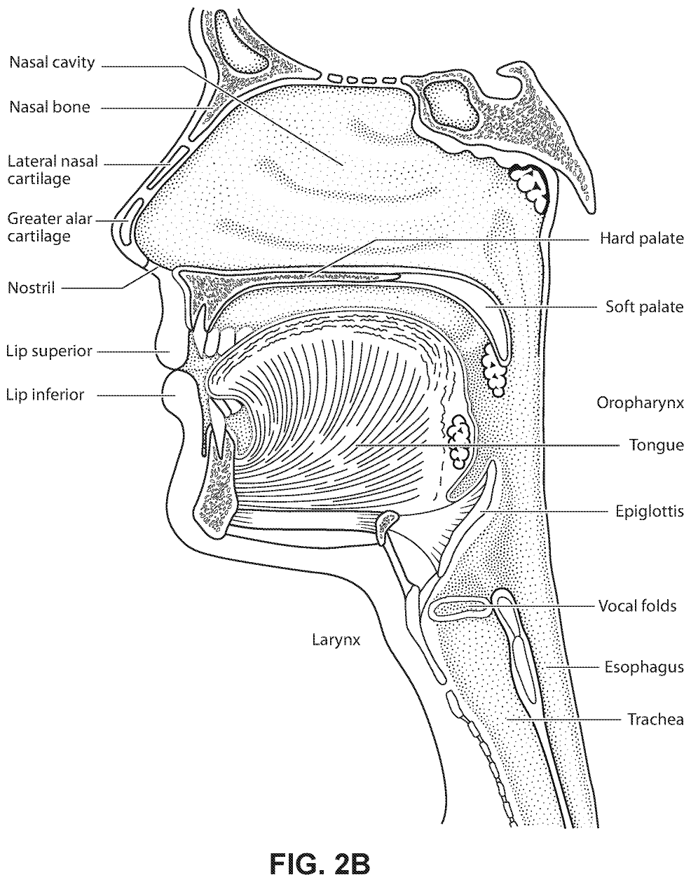

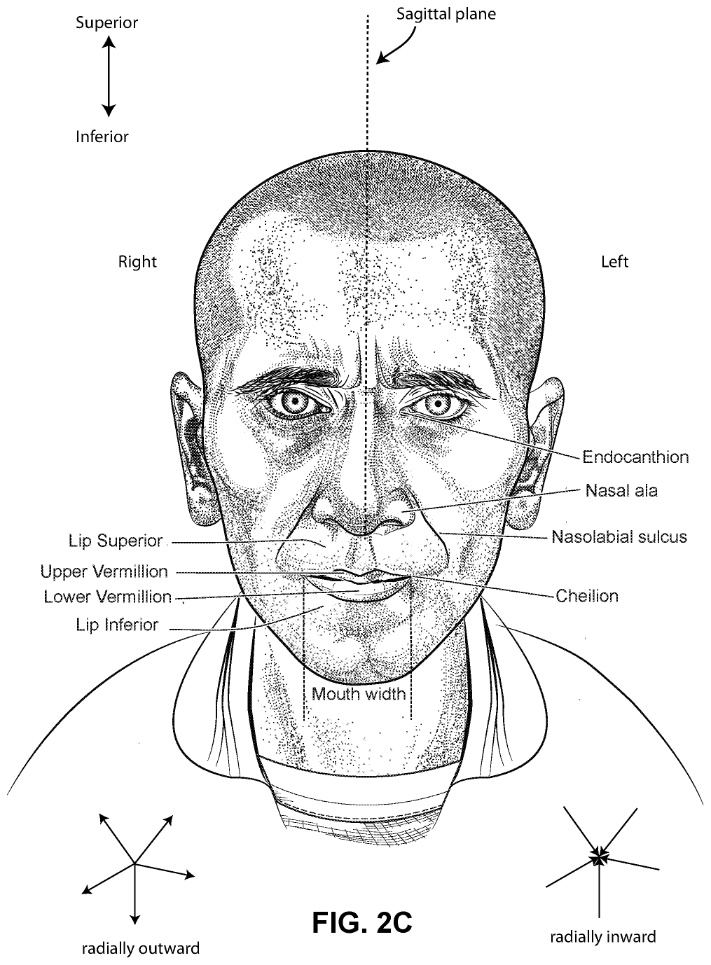

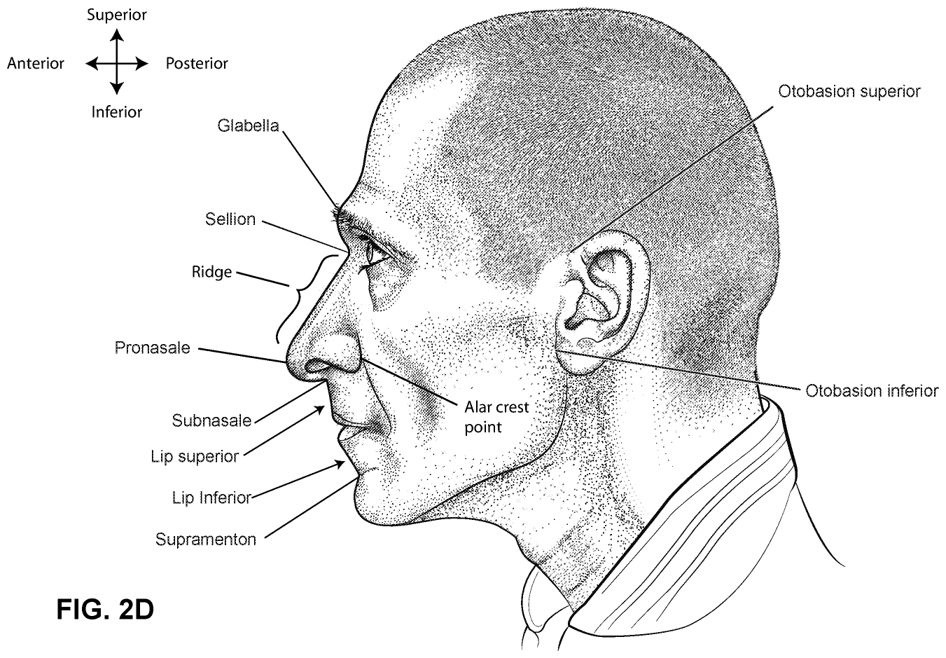

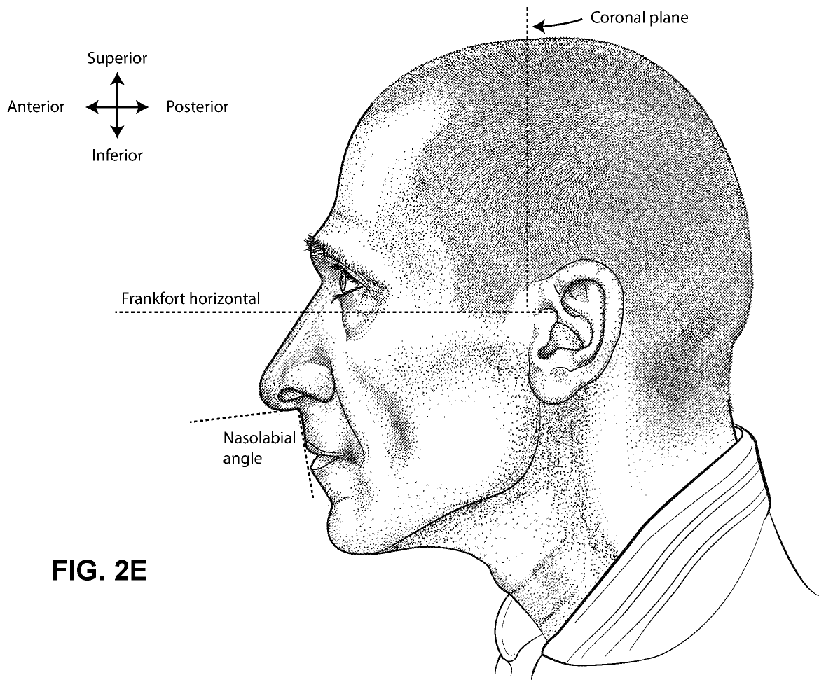

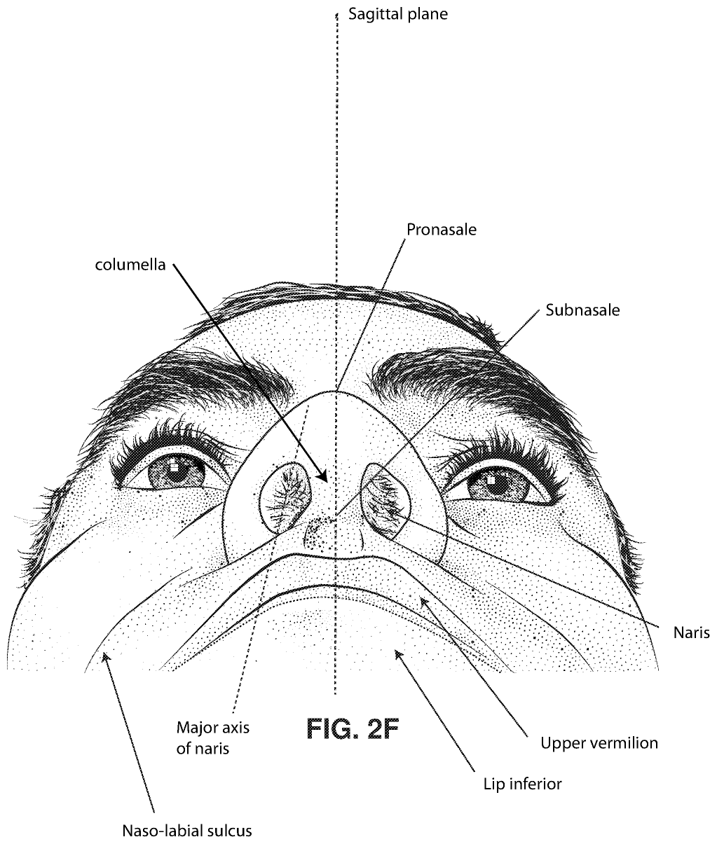

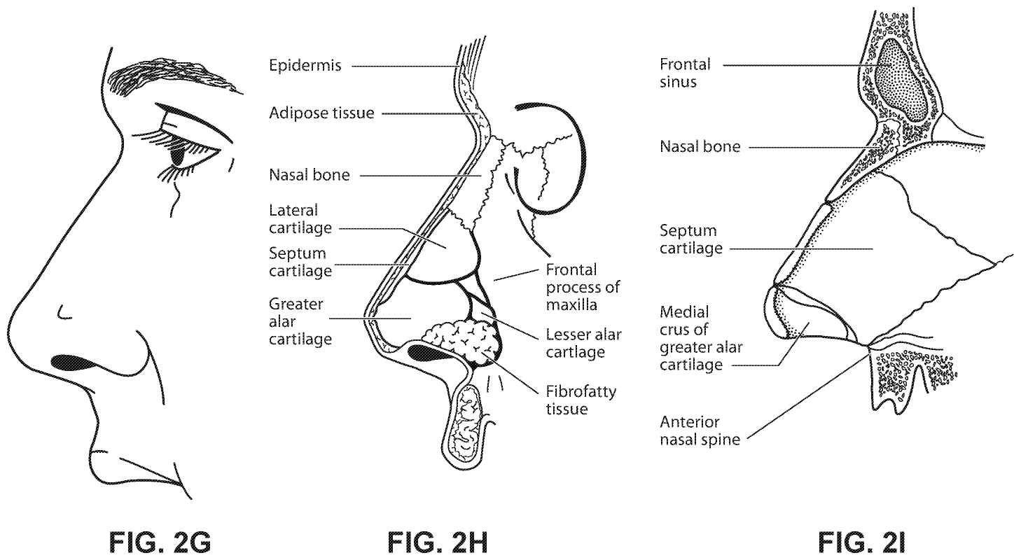

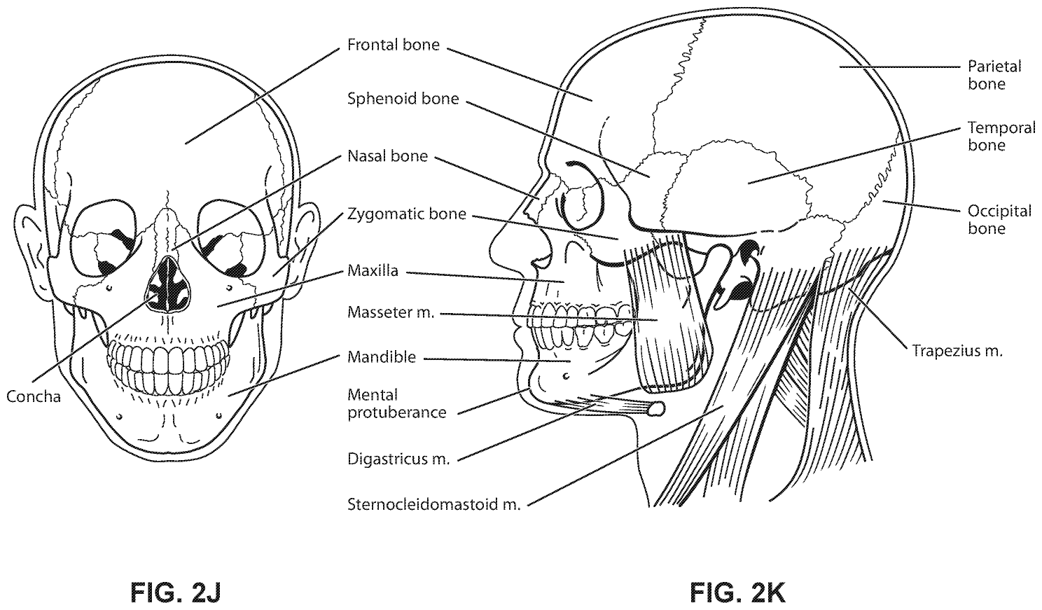

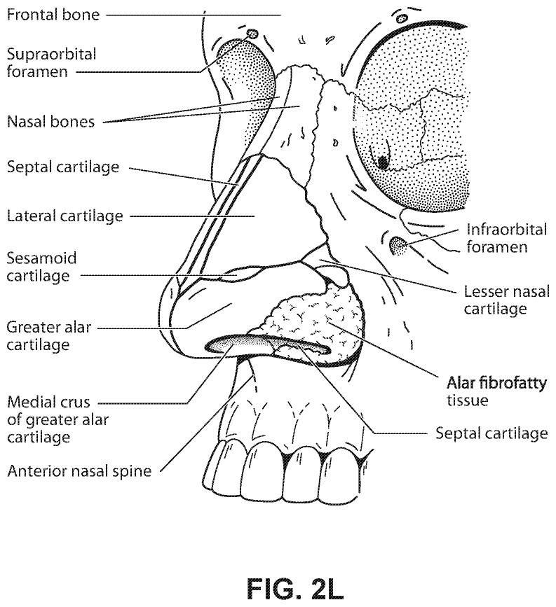

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideo trapezius.

FIG. 2L shows an anterolateral view of a nose.

### 3.3 PATIENT INTERFACE

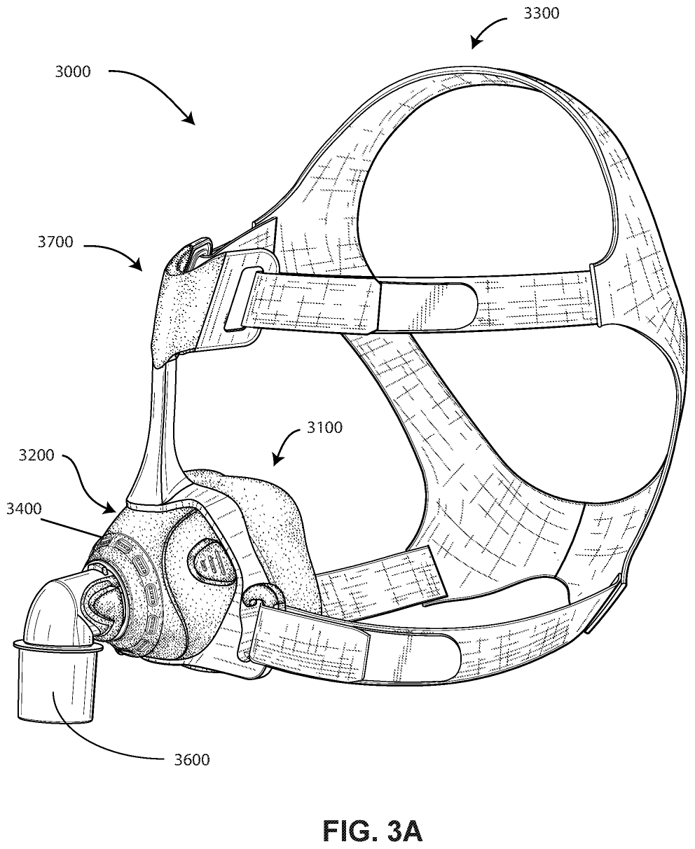

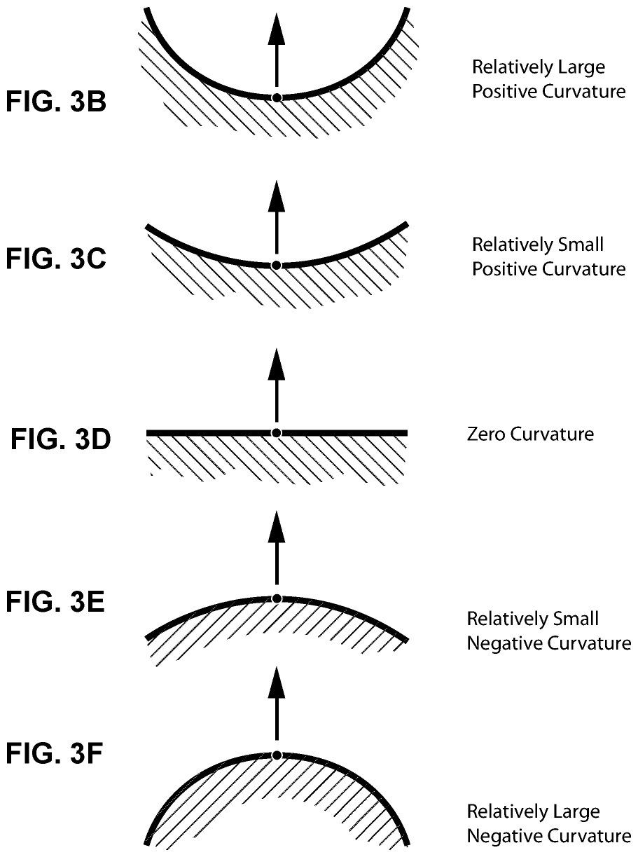

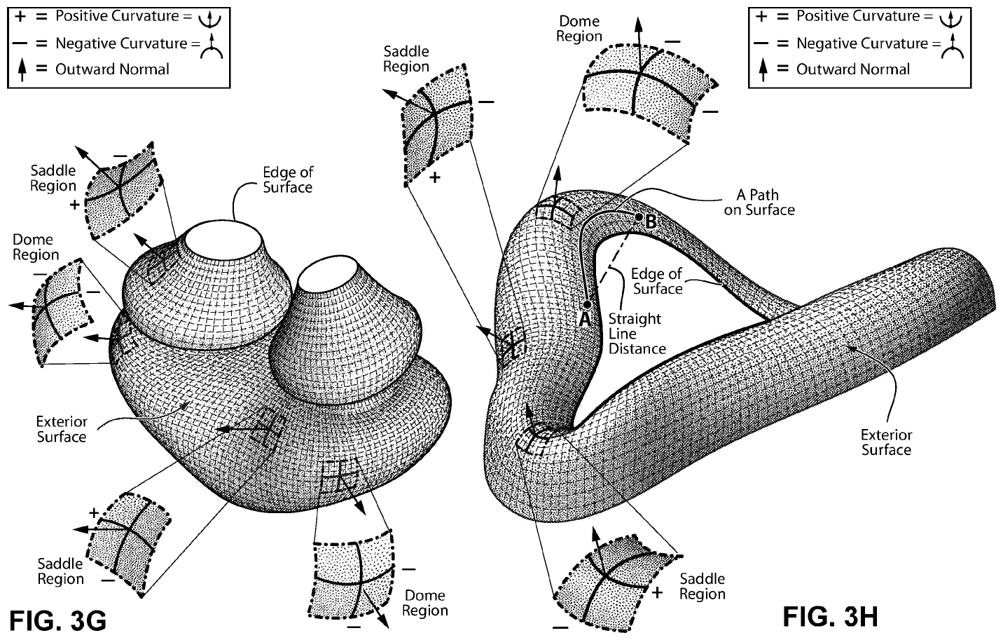

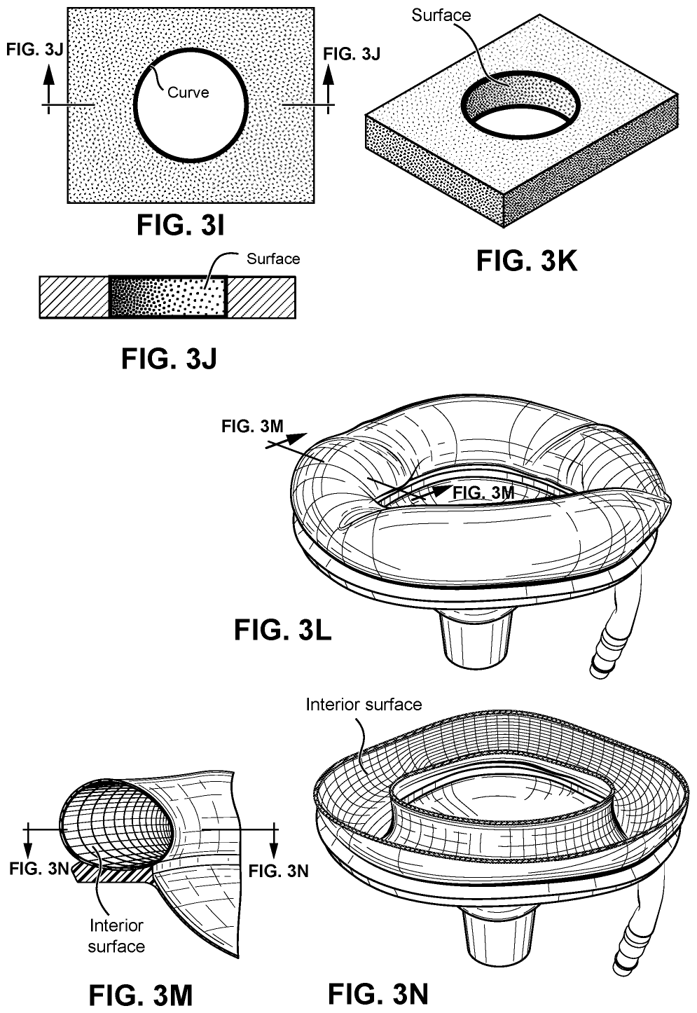

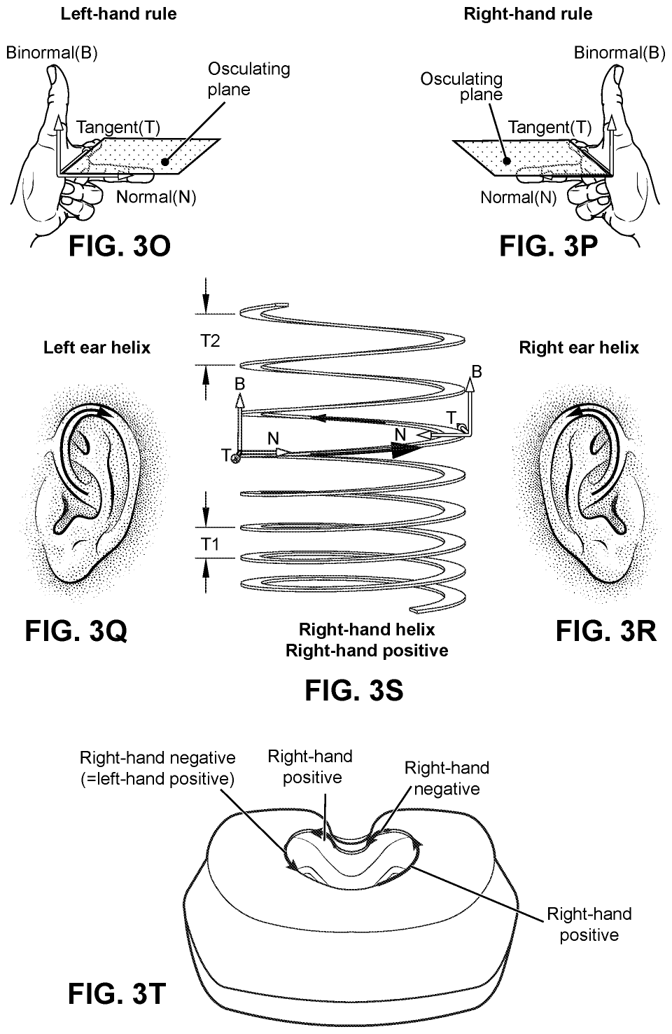

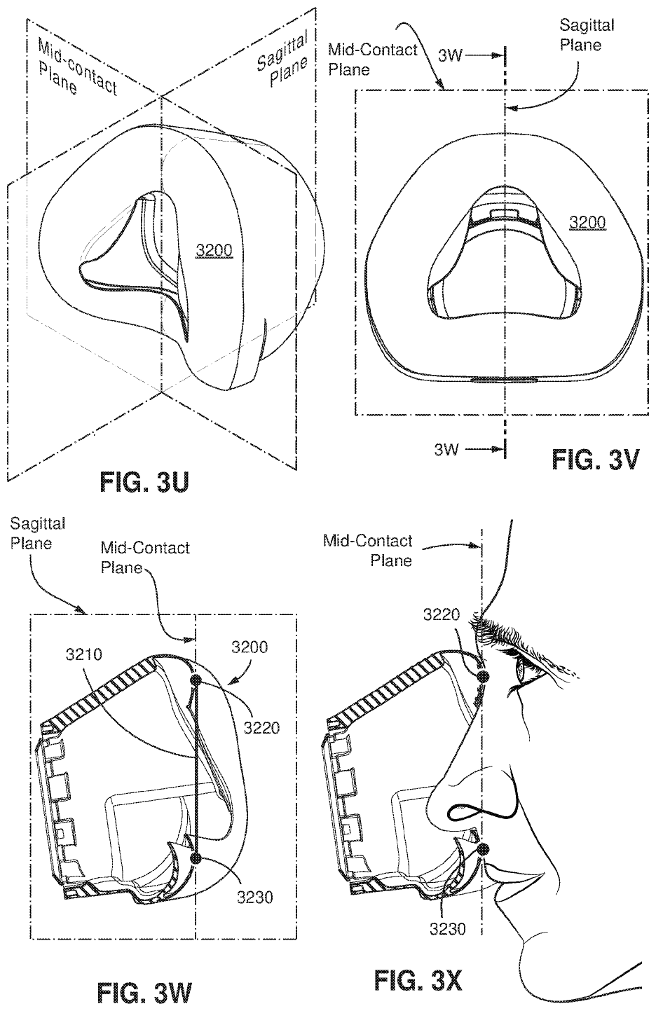

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

### 3.4 RPT DEVICE

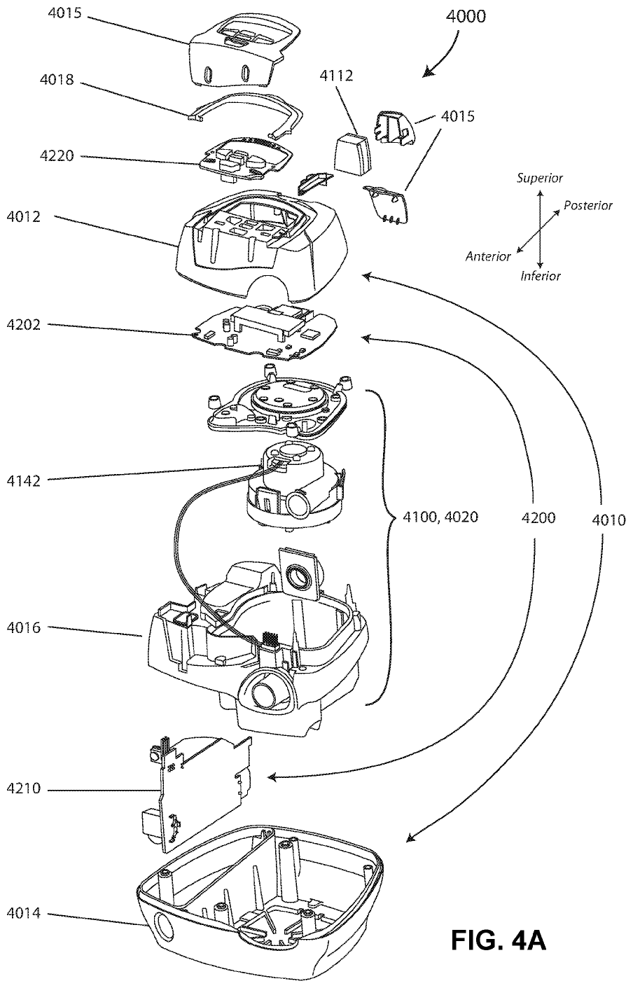

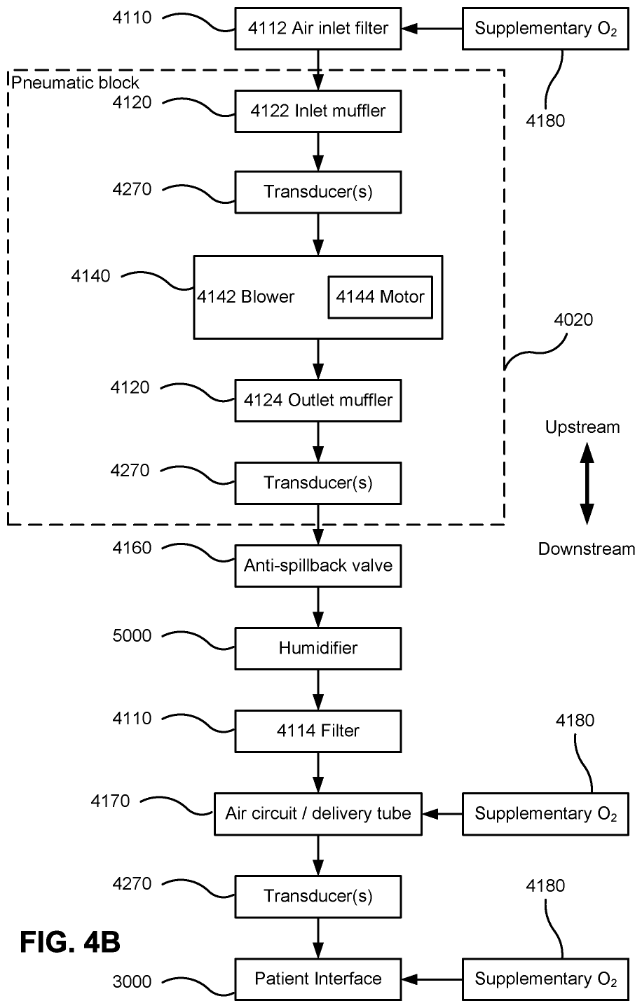

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 HUMIDIFIER

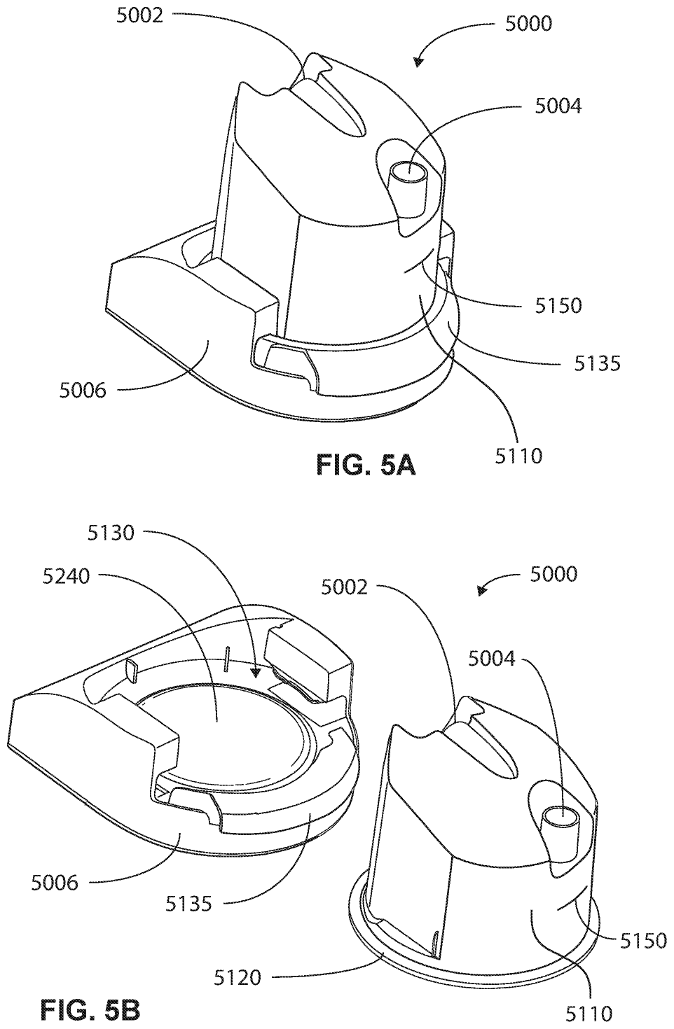

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 FURTHER FORMS OF PATIENT INTERFACE AND POSITIONING AND STABILISING STRUCTURE FOR PATIENT INTERFACES

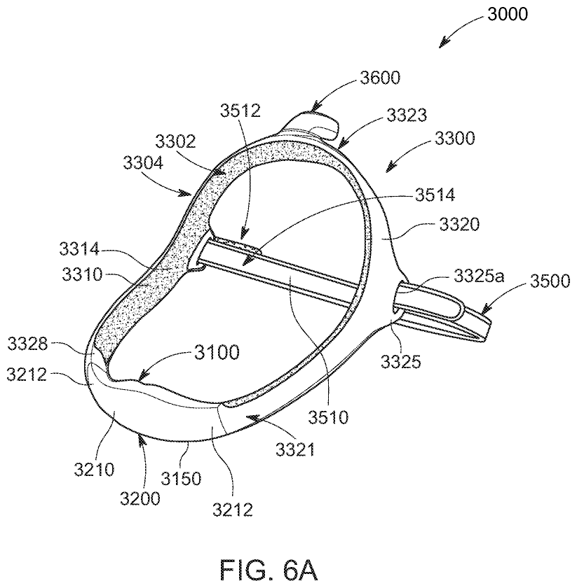

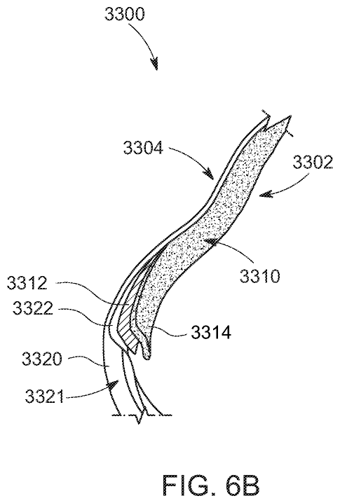

FIG. 6A shows a perspective view of a patient interface in accordance with one form of the present technology.

FIG. 6B shows an enlarged perspective view of a section of a positioning and stabilising structure in accordance with one form of the present technology.

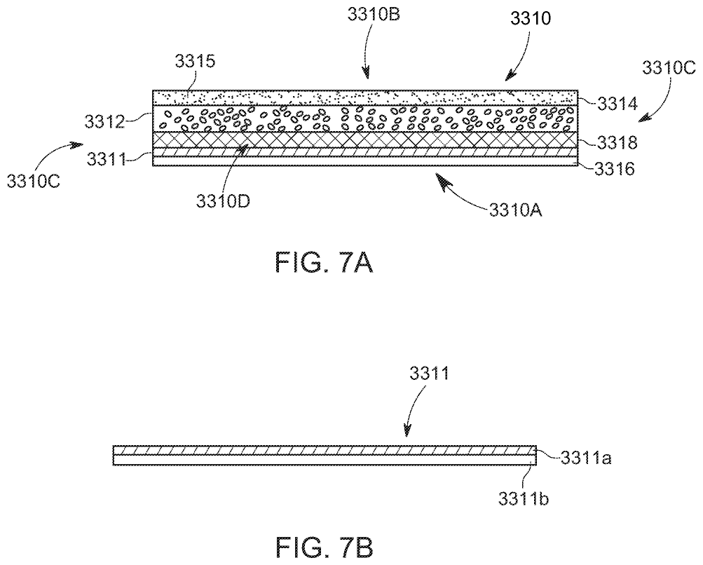

FIG. 7A shows a cross-sectional view through a section of a pad in accordance with one form of the present technology.

FIG. 7B shows a cross-sectional view through a section of an adhesive layer of a pad in accordance with one form of the present technology.

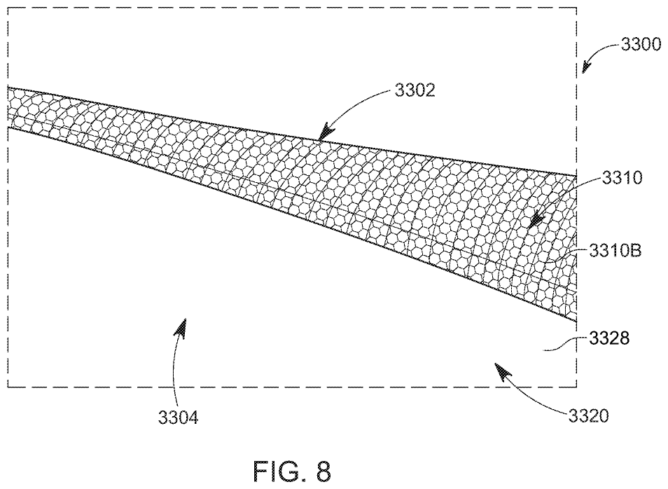

FIG. 8 shows an enlarged perspective view of a section of the positioning and stabilising structure in accordance with one form of the present technology, showing a joint between a conduit and a pad.

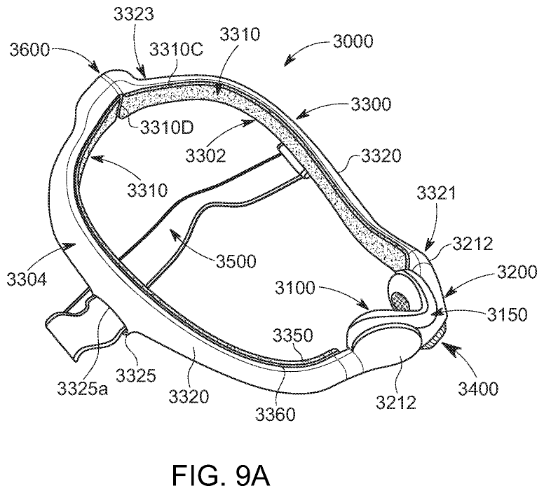

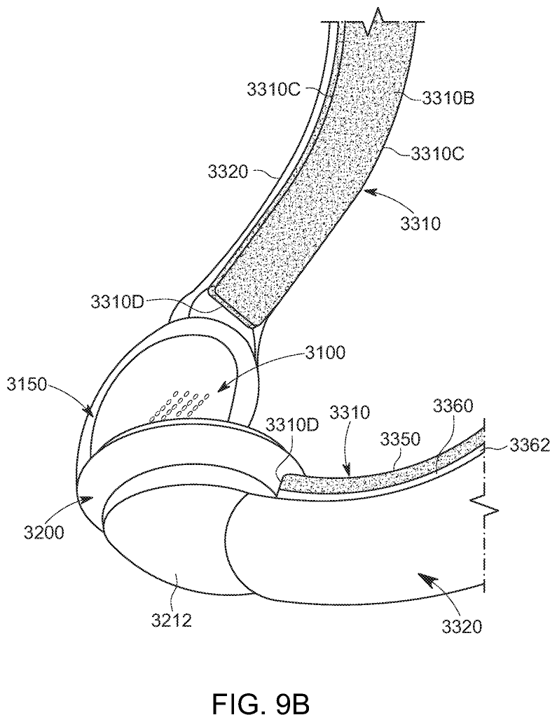

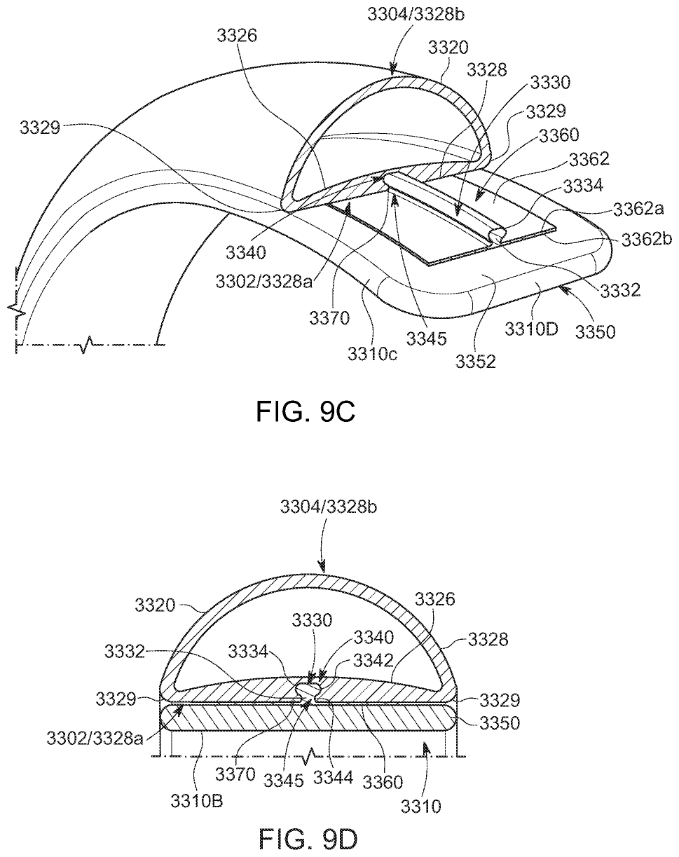

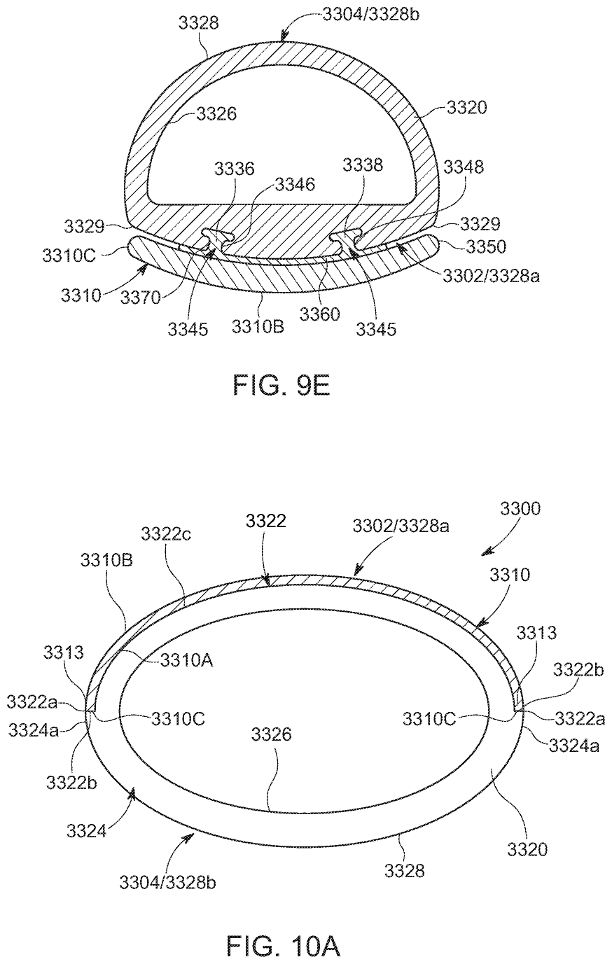

FIG. 9A shows a perspective view of a patient interface in accordance with one form of the present technology.

FIG. 9B shows an enlarged perspective view of a section of the patient interface of FIG. 9A.

FIG. 9C shows a cross-sectional view through a section of a positioning and stabilising structure in accordance with one form of the present technology, showing a cross-sectional view through a section of a conduit and not through a pad.

FIG. 9D shows a cross-sectional view through a section of the positioning and stabilising structure of FIG. 9C.

FIG. 9E shows a cross-sectional view through a section of a positioning and stabilising structure in accordance with one form of the present technology.

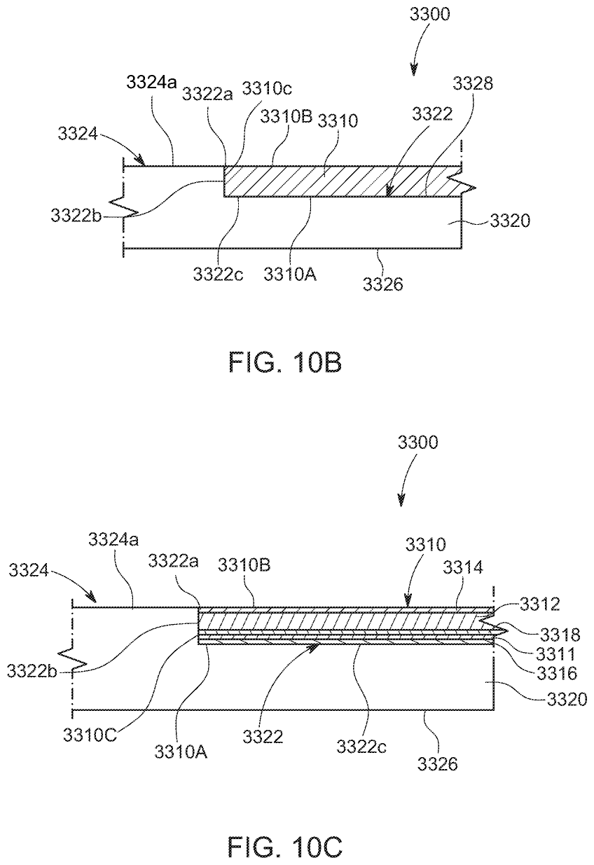

FIG. 10A shows a cross-sectional view through a section of a positioning and stabilising structure in accordance with one form of the present technology.

FIG. 10B shows an enlarged cross-sectional view of a section of the positioning and stabilising structure of FIG. 10A.

FIG. 10C shows the section of the positioning and stabilising structure of FIG. 10B which has the pad of FIG. 7A attached to the conduit, in accordance with one form of the present technology.

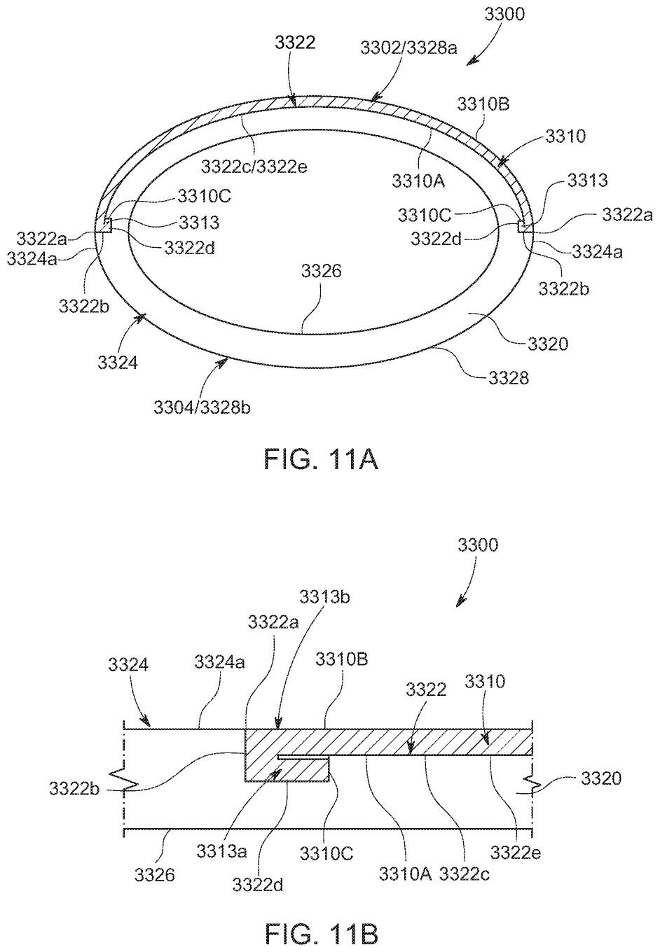

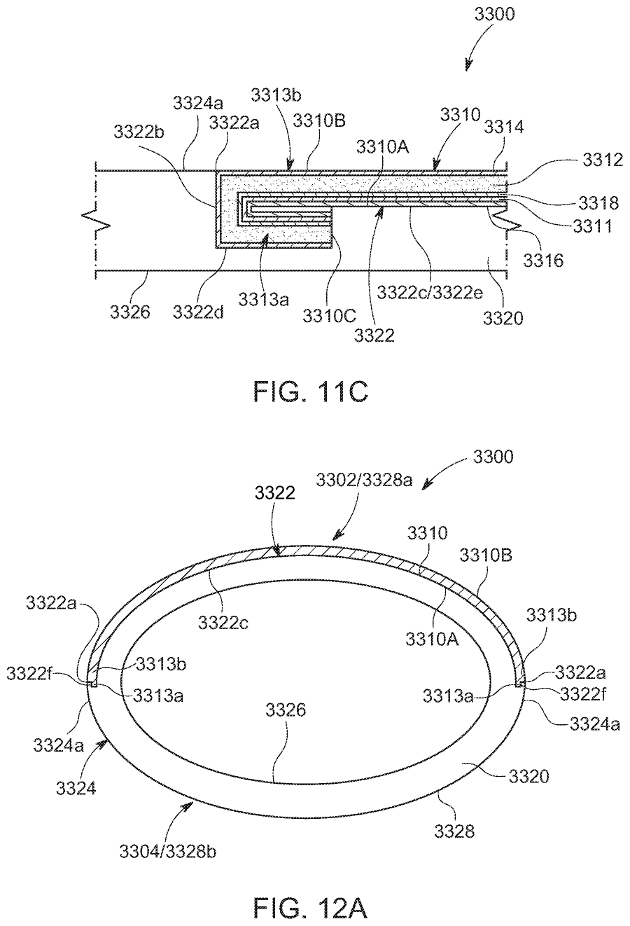

FIG. 11A shows a cross-sectional view through a section of a positioning and stabilising structure in accordance with one form of the present technology.

FIG. 11B shows an enlarged cross-sectional view of a section of the positioning and stabilising structure of FIG. 11A.

FIG. 11C shows the section of the positioning and stabilising structure of FIG. 11B which has the pad of FIG. 7A attached to the conduit, in accordance with one form of the present technology.

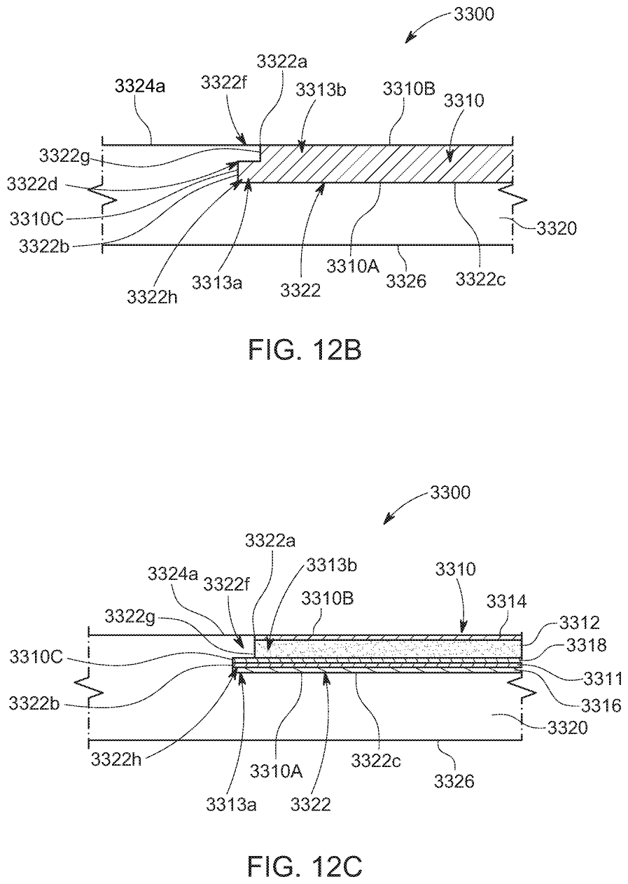

FIG. 12A shows a cross-sectional view through a section of a positioning and stabilising structure in accordance with one form of the present technology.

FIG. 12B shows an enlarged cross-sectional view of a section of the positioning and stabilising structure of FIG. 12A.

FIG. 12C shows the section of the positioning and stabilising structure of FIG. 12B which has the pad of FIG. 7A attached to the conduit, in accordance with one form of the present technology.

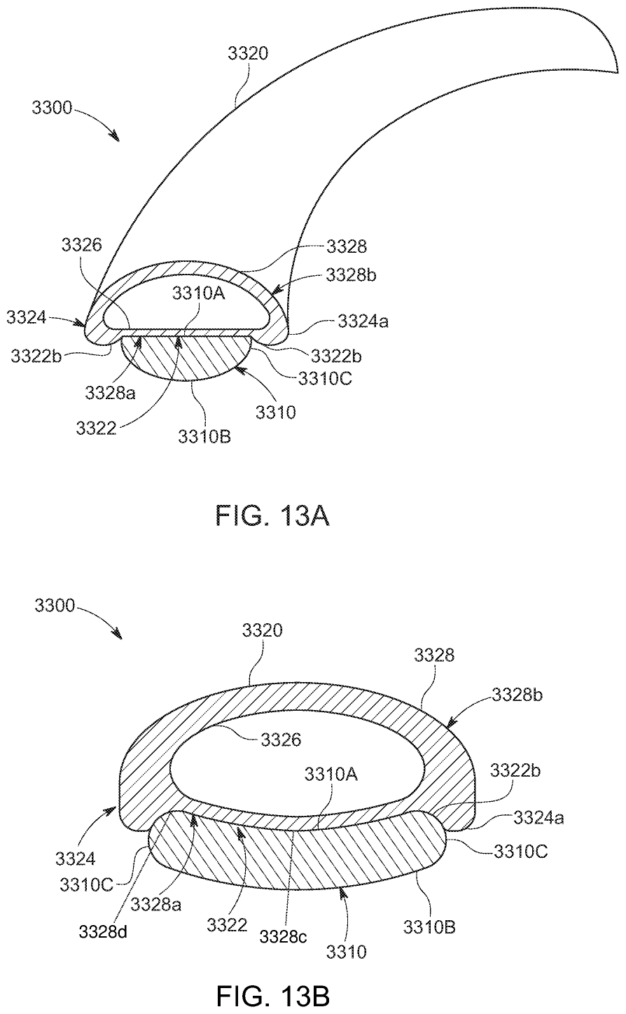

FIG. 13A shows a cross-sectional view through a section of a positioning and stabilising structure, in accordance with one form of the present technology.

FIG. 13B shows a cross-sectional view through a section of a positioning and stabilising structure, in accordance with one form of the present technology.

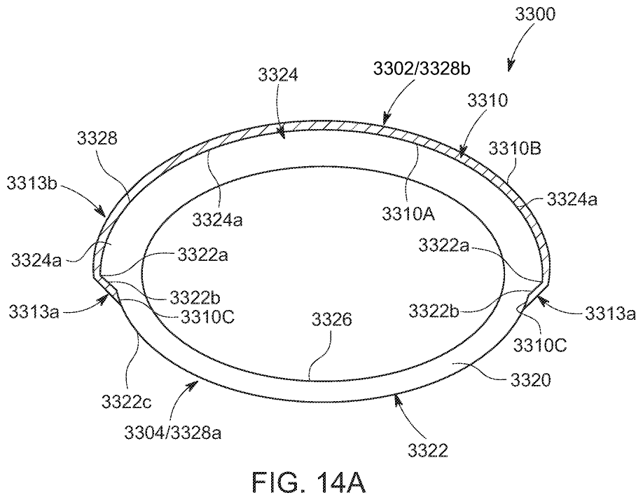

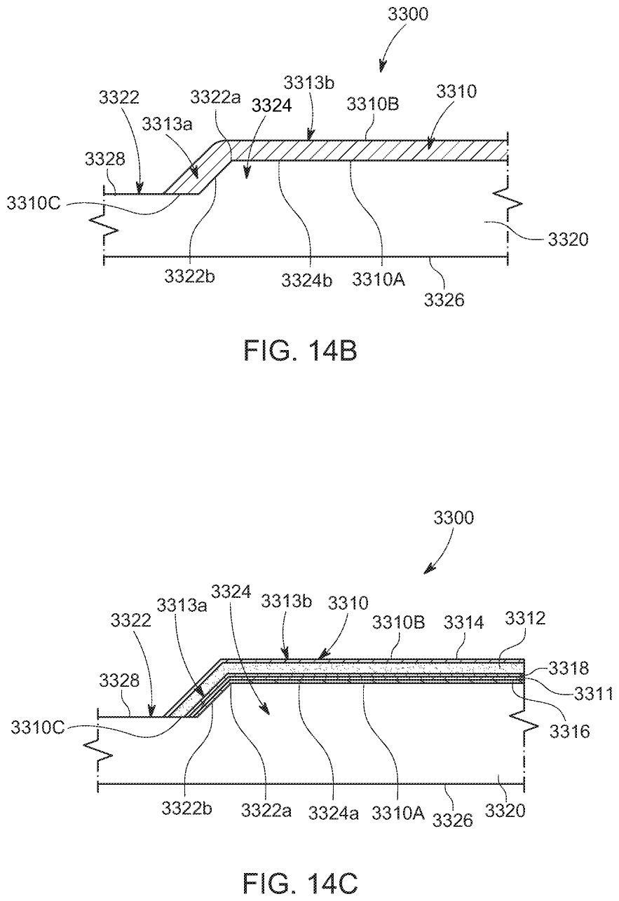

FIG. 14A shows a cross-sectional view through a section of a positioning and stabilising structure in accordance with one form of the present technology.

FIG. 14B shows an enlarged cross-sectional view of a section of the positioning and stabilising structure of FIG. 14A.

FIG. 14C shows the section of the positioning and stabilising structure of FIG. 14B which has the pad of FIG. 7A attached to the conduit, in accordance with one form of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 THERAPY

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 RESPIRATORY THERAPY SYSTEMS

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 4.3 PATIENT INTERFACE

A non-invasive patient interface 3000 in accordance with one aspect of the present technology is illustrated in FIG. 3A, and comprises the following functional aspects: a seal-forming structure 3100, a plenum chamber 3200, a positioning and stabilising structure 3300, a vent structure 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

FIGS. 6A and 9A illustrate a patient interface 3000 in accordance with forms of the present technology. The patient interface 3000 comprises a seal-forming structure 3100, a plenum chamber 3200, and a positioning and stabilising structure 3300. The positioning and stabilising structure 3300 comprises a conduit 3320 and a pad 3310 attached to the conduit 3320. In the illustrated forms, the conduit 3320 is configured to permit delivery of a flow of air at a therapeutic pressure into the plenum chamber 3200.

In some forms, the conduit may comprise a plurality of conduits 3320, e.g. a pair of conduits 3320, therefore reference herein to “conduit” may include reference to “conduits”. In some forms, the pad 3310 may comprise a plurality of pads 3310, e.g. at least two pads 3310, therefore reference herein to “pad” may include reference to “pads”

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH₂O with respect to ambient.

The patient interface 3000 in accordance with one form of the present technology is constructed and arranged to be able to provide a supply of air at a positive pressure of at least 10 cmH₂O with respect to ambient.

The patient interface 3000 in accordance with one form of the present technology is constructed and arranged to be able to provide a supply of air at a positive pressure of at least 20 cmH₂O with respect to ambient.

### 4.3.1 Plenum Chamber

A patient interface 3000 according to some examples of the present technology comprises a plenum chamber 3200 pressurisable to a therapeutic pressure of at least 6 cmH₂O above ambient air pressure. The plenum chamber 3200 may receive a flow of air at the therapeutic pressure for breathing by a patient.

The plenum chamber 3200 in some forms of the present technology is at least partially provided by a cushion module 3150 of the patient interface 3000.

As is perhaps best illustrated in FIG. 6A, the cushion module 3150 according to some examples may comprise a frame portion 3210 and the seal-forming structure 3100.

The plenum chamber 3200 may be at least partially formed by both the frame portion 3210 and the seal-forming structure 3100. The frame portion 3210 may support the seal-forming structure 3100 in position against the patient's face in use. The frame portion 3210 and seal-forming structure 3100 may together partially enclose a volume of space which in use has air therein pressurised above atmospheric pressure, forming the plenum chamber 3200.

In particular, the frame portion 3210 may at least partially define part of the plenum chamber 3200 pressurisable to a therapeutic pressure of at least 6 cmH₂O above ambient air pressure.

The frame portion 3210 may comprise one or more laterally projecting connection portions 3212 configured to connect to the headgear conduit system 3300. The laterally projecting connection portions 3212 may also partially form the plenum chamber 3200 along with other portions of the cushion module 3150, that is the laterally projecting connection portions 3212 may be configured to each contain a volume, where the volume inside the laterally projecting connection portions 3212 is part of the volume inside the plenum chamber 3200.

The seal-forming structure 3100 may be provided to the frame portion 3210 and may at least partially form the plenum chamber 3200. The seal-forming structure 3100 may be connected to the frame portion 3210, either permanently connected or removably connected.

In certain forms, the plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200.

In some forms, the plenum chamber 3200 is formed from a single homogeneous piece of material. In some forms, the plenum chamber 3200 may be formed from a homogenous piece of material fitted with connectors formed from another material. In other forms, the plenum chamber 3200 is constructed from a plurality of materials, for example one material may be used to form the frame portion 3210 and another material may be used to form the seal-forming structure 3100, with the plenum chamber 3200 comprising at least a part of both the frame portion 3210 and the seal-forming structure 3100.

In certain forms of the present technology, the plenum chamber 3200 does not cover the eyes of the patient in use. In other words, the eyes are outside the pressurised volume defined by the plenum chamber 3200. Such forms tend to be less obtrusive and/or more comfortable for the wearer, which can improve compliance with therapy.

In certain forms of the present technology, the plenum chamber 3200 is constructed from a transparent material, e.g. silicone, a thermoplastic elastomer, a transparent polycarbonate or the like. For example, the majority of the cushion module 3150 can be formed from silicone. In particular, the frame portion 3210 and seal-forming structure 3100 are both formed from silicone in those examples. The use of a transparent material can reduce the obtrusiveness of the patient interface 3000, and help improve compliance with therapy. The use of a transparent material can aid a clinician to observe how the patient interface 3000 is located and functioning.

In certain forms of the present technology, the plenum chamber 3200 is constructed from a translucent material. The use of a translucent material can reduce the obtrusiveness of the patient interface 3000, and help improve compliance with therapy.

In the exemplary forms of the technology, the frame portion 3210 may be flexible, for example it may be formed from a material having a relatively low modulus of elasticity. The seal-forming structure 3100 may also be flexible, for example formed from the same material, or another material having a relatively low modulus of elasticity. In some examples, the frame portion 3210 and seal-forming structure 3100 are integrally formed and may be formed from the deformable material. The frame portion 3210 may be formed from an elastomeric material, such as silicone. The seal-forming structure 3100 may be formed from the elastomeric material. The frame portion 3210 and seal-forming structure 3100 may comprise one piece. In these examples, the frame portion 3210 and seal-forming structure 3100 are moulded together as a single part formed from an elastomeric material, e.g. silicone. The seal-forming structure 3100 and the frame portion 3210 (or at least a majority of the frame portion 3210) may be formed (e.g. constructed, moulded or the like) together from a single homogenous piece of a deformable material, such as an elastomeric material, e.g. silicone. The frame portion 3210 and the seal-forming structure 3100 may be of unitary construction.

In other examples of the technology the seal-forming structure 3100 may be removably connected to the frame portion 3210. The seal-forming structure 3100 may be connected to the frame portion 3210 by a soft-to-soft, soft-to-hard or a hard-to-hard connection.

In the form of the present technology, the plenum chamber 3200 comprises an inlet (not shown) configured to receive a flow of air at the therapeutic pressure for breathing by the patient. In some forms, the plenum chamber 3200 comprises a pair of inlets, configured to permit fluid communication with an interior of the plenum chamber 3200. For example, the plenum chamber 3200 comprises a pair of lateral inlets. However, the inlets may be formed in any other suitable location on the plenum chamber, e.g. on an anterior portion of the plenum chamber 3200. In some forms, each laterally projecting connection portion 3212 comprises the inlet in the form of an opening which is configured to be in fluid communication with an interior of the plenum chamber 3200.

In some examples, at least one rigidiser may be provided to the frame portion 3210 to make it more rigid (while still being flexible). In some examples, the frame portion 3210 and the seal-forming structure 3100 are formed from a material having a relatively low modulus of elasticity (e.g. silicone, TPE or the like) and the frame portion 3210 comprises the rigidiser(s). In particular, the patient interface 3000 or cushion module 3150 may comprise the rigidiser(s). The rigidiser(s) may be provided to the frame portion 3210. The rigidiser(s) may be configured to rigidise the frame portion 3210. The rigidiser(s) may be configured to resist deformation of the frame portion 3210. The rigidiser(s) may be configured to provide support to the frame portion 3210.

The rigidiser(s) may be provided by one or more of: a thickened region, a structure or component which is rigid relative to the frame portion and which is one of removably attached, permanently attached, and integrally formed with the frame portion 3210.

### 4.3.2 Seal-Forming Structure

A patient interface 3000 according to some examples of the present technology comprises a seal-forming structure 3100 configured to form a seal with a region of the patient's face surrounding an entrance to the patient's airways. The seal-forming structure 3100 may be configured to maintain said therapeutic pressure in the plenum chamber 3200 throughout the patient's respiratory cycle in use.

In some forms, the seal-forming structure 3100 is configured to form a seal with or around portions of the patient's nose in use. For example, the seal-forming structure 3100 is configured to form a seal in use with the underside of the patient's nose, around the patient's nares. In the illustrated example, the patient interface 3000 is in the form of a nasal mask.

The seal-forming structure 3100 may comprise at least one hole, or a pair of holes, neither of which are shown in the figures, but which may be configured to allow a flow of air at therapeutic pressure to be delivered to the patient's nares. Each hole aligns in use with a respective nare of the patient. In some examples, the at least one hole is formed in a central portion of the seal-forming structure 3100.

In other forms which are not illustrated, the seal-forming structure 3100 may be configured to form a seal in use with the other parts of patient's face, e.g. with or around portions of the patient's nose and with or around portions of the patient's mouth. Therefore, it is to be appreciated that in some forms the patient interface 3000 may be a full face or oro-nasal mask and is not limited to a nasal mask.

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.2.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.2.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.2.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.2.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.2.5 Forehead Region

In one form, the seal-forming structure that forms a seal in use on a forehead region of the patient's face. In such a form, the plenum chamber may cover the eyes in use.

### 4.3.2.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.2.7 Textile Seal

In some forms, the seal-forming structure 3100 comprises a textile seal.

That is, the seal-forming structure 3100 comprises a layer of textile material or fabric which may seal with the region of the patient's face surrounding the entrance to the patient's airways.

### 4.3.3 Positioning and Stabilising Structure

The seal-forming structure 3100 of the patient interface 3000 of the present technology may be held in the sealing position in use by the positioning and stabilising structure 3300.

In some forms, at least part of the positioning and stabilising structure 3300 is provided by the conduit 3320. The arrangement of the conduit 3320 which is positioned, in use, on the patient's head and/or face, may be referred to as a “headgear conduit system”, or the likes thereof, as explained earlier.

In some forms, the positioning and stabilising structure 3300 comprises at least one strap 3500. As illustrated, in some forms, the at least one strap 3500 may be attached to the conduit 3320. However, in other forms which are not shown in the figures, the at least one strap 3500 may be attached to the pad 3310.

It is to be appreciated that in yet other forms, also not shown in the figures, at least one headgear strap may be attached to the plenum chamber 3200 and/or seal-forming structure 3100. This may be in addition to the headgear strap 3500, described above. For example, a full-face mask or an oro-nasal mask may comprise the headgear strap 3500 attached to the conduit 3320 and/or pad 3310 of the positioning and stabilising structure 3300, and another headgear strap attached to a portion of the plenum chamber 3200.

In one form the positioning and stabilising structure provides a retention force at least sufficient to overcome the effect of the positive pressure in the plenum chamber 3200 to lift off the face.

In one form the positioning and stabilising structure provides a retention force to overcome the effect of the gravitational force on the patient interface 3000.

In one form the positioning and stabilising structure provides a retention force as a safety margin to overcome the potential effect of disrupting forces on the patient interface 3000, such as from tube drag, or accidental interference with the patient interface.

In one form of the present technology, a positioning and stabilising structure is provided that is configured in a manner consistent with being worn by a patient while sleeping. In one example the positioning and stabilising structure has a low profile, or cross-sectional thickness, to reduce the perceived or actual bulk of the apparatus.

In one example, the positioning and stabilising structure comprises at least one strap 3500. The strap 3500 may have a rectangular cross-section. The strap 3500 may be a substantially flat strap 3500. However, the at least one strap 3500 may have any other suitably shaped cross-section.

In one form of the present technology, a positioning and stabilising structure is provided that is configured so as not to be too large and bulky to prevent the patient from lying in a supine sleeping position with a back region of the patient's head on a pillow.

In one form of the present technology, a positioning and stabilising structure is provided that is configured so as not to be too large and bulky to prevent the patient from lying in a side sleeping position with a side region of the patient's head on a pillow.

In one form of the present technology, a positioning and stabilising structure is provided with a decoupling portion located between an anterior portion of the positioning and stabilising structure, and a posterior portion of the positioning and stabilising structure. The decoupling portion does not resist compression and may be, e.g. a flexible or floppy strap 3500. The decoupling portion is constructed and arranged so that when the patient lies with their head on a pillow, the presence of the decoupling portion prevents a force on the posterior portion from being transmitted along the positioning and stabilising structure and disrupting the seal.

In one form of the present technology, the at least one strap 3500 may be constructed from a laminate of a fabric patient-contacting layer, a foam inner layer (not shown) and a fabric outer layer 3510. In one form, the foam is porous to allow moisture, (e.g., sweat), to pass through the strap 3500. In one form, the fabric outer layer 3510 comprises loop material portion to engage with a hook material portion, e.g Velcro™

In some forms, the conduits 3320 may comprise at least one connector 3325 to facilitate attachment of the strap 3500 to the conduits 3320. For example, as illustrated in FIGS. 6A and 9A, the connector 3325 may comprise a slot 3325a configured to receive a portion of the strap 3500. The connector 3325 may take the form of a tab extending in a posterior direction from the main body of the conduit 3320.

In other forms not illustrated in the figures, the pad(s) 3310 may cover at least a portion of the connector(s) 3325, e.g. a patient facing side of the connector(s) 3325.

In yet other forms not illustrated, the pad(s) 3310 may comprise at least one connector to facilitate attachment of a strap 3500 to the pad(s) 3310. A portion of the pad 3310 may be configured to provide the connector, for example in the form of a tab extending in a posterior direction from the main body of the pad 3310.

The strap 3500 length may be adjusted to adjust the headgear forces provided by the positioning and stabilising structure. For example, at least one portion 3512 of the strap 3500 comprises one of the loop material portion and the hook material portion described above, and another portion 3514 of strap 3500 comprises the other one of the loop material portion and the hook material portion.

In certain forms of the present technology, a positioning and stabilising structure comprises a strap 3500 that is extensible, e.g. resiliently extensible. For example the strap 3500 may be configured in use to be in tension, and to direct a force to draw a seal-forming structure 3100 into sealing contact with a portion of a patient's face. In an example the strap 3500 may be configured as a tie.

In certain forms of the present technology, a positioning and stabilising structure comprises a strap 3500 that is bendable and e.g. non-rigid. An advantage of this aspect is that the strap 3500 is more comfortable for a patient to lie upon while the patient is sleeping.

In certain forms of the present technology, a positioning and stabilising structure comprises a strap 3500 constructed to be breathable to allow moisture vapour to be transmitted through the strap 3500.

In certain forms of the present technology, a patient interface is provided comprising more than one positioning and stabilising structure, each being configured to provide a retaining force to correspond to a different size and/or shape range. For example the system may comprise one form of positioning and stabilising structure suitable for a large sized head, but not a small sized head, and another suitable for a small sized head, but not a large sized head.

### 4.3.3.1 Positioning and Stabilising Structure Comprising Conduit and Pad

FIGS. 6A to 14C illustrate forms of the positioning and stabilising structure 3300 which is configured to facilitate delivery of pressurised breathable gas to the interior of the plenum chamber 3200, and configured to position and stabilise the seal-forming structure 3100 on the patient's face in use.

Therefore, the conduit 3320 defines a flow path for the passage of pressurised air therethrough. In addition, the conduit(s) 3320, and the pad(s) 3310 attached thereto, form part of the positioning and stabilising structure 3300 which holds the seal-forming structure in a sealing position, wherein the conduit(s) 3320 and the pad(s) 3310 are positioned, in use, on the patient's head and/or face. The pad(s) 3310 is/are attached to the conduit(s) 3320 such that at least part of the pad(s) 3310 contact the patient's head and/or face in use.

Although not shown in the figures, the positioning and stabilising structure 3300 may be configured to be positioned such that in use, parts of the conduits 3320 and pads 3310 extend along anterior and lateral portions of the patient's face and/or head, e.g. across the patient's cheeks and temples, while other parts extend, in use, over superior regions of the patient's face and/or head, e.g across the patient's temple and over the top of the head. The conduits 3320 may be configured to fluidly connect the plenum chamber 3200, positioned anterior to the patient's nose/mouth region in use, to a connection port 3600, which is positioned superior to the patient's otobasion superior in use. For example, the connection port 3600 may be positioned on top of the patient's head in use.

As illustrated in FIG. 6A, in one form, the positioning and stabilising structure 3300 comprises a single pad 3310 and a pair of conduits 3320. However, as mentioned, in other forms such as the form shown in FIG. 9A, the positioning and stabilising structure 3300 may comprise a plurality of pads 3310, e.g. a pair of pads 3310 which are each attached to one of the pair of conduits 3320.

In some forms, the positioning and stabilising structure 3300 may comprise a headgear member having a form other than a conduit. For example, the headgear member may comprise one or more straps and/or rigidising elements. While the foregoing description of exemplary pads 3310 is described in relation to forms of the technology in which the positioning and stabilising structure 3300 comprises a headgear member in the form of one or more conduits, it should be understood that the description also applies to forms in which the positioning and stabilising structure 3300 comprises another form of headgear member.

### 4.3.3.2 Pads

The pad 3310 comprises a conduit facing surface 3310A, an opposing patient facing surface 3310B, opposing lateral edges 3310C and opposing end edges 3310D. The conduit facing surface 3310A is positioned on the conduit 3320 such that the patient facing surface 3310B faces towards, and may contact, the patient's head and/or face in use. Lateral edges 3310C may also be referred to as “sides”, “side edges” or the like thereof, and end edges may also be referred to as “ends” or the like thereof. The lateral edges are generally more elongate than the end edges.

The pad 3310 may be formed in any suitable shape or size. In certain forms, as illustrated, the length of the pad 3310 (i.e. the distance between end edges 3310D) may be greater than the width of the pad 3310 (i.e. the distance between lateral edges 3310C), which may be greater than the thickness of the pad 3310 (i.e. the distance between the inner surface 3310A and the outer surface 3310B).

In some forms, when viewing the pad 3310 in a direction perpendicular to the end edge 3310D, the pad 3310 may have a substantially flat lateral edge 3310C, as shown in FIGS. 10A to 11C. In some forms, the pad 3310 may have convex-shaped lateral edges, as shown in FIGS. 9D and 9E. Therefore, the lateral edges 3310C may be rounded or curved in some forms. Similarly, the end edges 3310D may be convex-shaped, i.e. rounded or curved, as shown in FIG. 9C.

The pad may comprise joint regions which are junctions between the respective surfaces 3310A, 3310B and edges 3310C, 3310D. In some forms, these joint regions may be convex-shaped, i.e. rounded or curved, as shown in FIGS. 9D and 9E. In some forms, these joint regions may not be rounded or curved, as shown in FIGS. 10A to 11C. These joint regions may be sharp or rough compared to a smoother, more rounded joint.

In general terms, the conduit 3320 comprises a patient facing side 3302 which includes surfaces of the conduits 3320 which face in a direction extending towards the patient's skin in use, and a non-patient facing side 3304 which includes the surfaces that face in a direction extending away from the patient's skin in use.

As illustrated, in certain forms, the pad 3310 is attached to at least a portion of the conduit 3320. The pad 3310 may be attached to an outer surface 3328 of the patient facing side 3302 of the conduit(s) 3320. The pad(s) may cover portions of the conduit(s) 3320 which would, but for the presence of the pads, contact the patient's head and/or face, in use, including portions of the conduit(s) 3320 that, but for the presence of the pads, might potentially contact the patient's head and/or face when the patient moves their head into different positions, for example, when sleeping, or when donning or doffing the patient interface 3000. Therefore, the pad(s) 3310 is/are positioned on the conduit(s) 3320 such that they contact the patient's skin and/or hair in use instead of the conduit 3320, which may improve patient comfort.

In some forms, as shown in FIGS. 6A to 14C, the outer surface 3328 of the conduit 3320 may comprise a first outer surface region 3328a and an adjacent second outer surface region 3328b. The first outer surface region 3328a is on the patient facing side of the conduit 3320 and the second outer surface region 3328b is on the non-patient facing side of the conduit 3320.

In some forms, as shown in FIGS. 6A to 13B, the pad 3310 may be positioned on the first outer surface region 3328a. In this example, the first outer surface region 3328a may be located on the patient facing side 3302 of the conduit 3320.

In some forms, as shown in FIGS. 14A to 14C, the pad 3310 may be positioned on the second outer surface region 3328b. In this example, the second outer surface region 3328b may be located on the patient facing side 3302 of the conduit 3320.

It is to be appreciated that in other forms which are not shown, the pad 3310 may be positioned on both parts of the first outer surface region 3328a and the second outer surface region 3328b. For example, the pad 3310 may cover the patient facing side 3302 and parts of the non-patient facing side 3304.

The pad 3310 may be attached to the conduits 3320 using any suitable method known to one skilled in the art. In some forms, the pad 3310 is attached to the conduit 3320 using a substrate layer 3316, e.g. a layer of semi-cured silicone. This aspect of certain forms of the current technology is discussed in more detail further below.

In other forms, the pad 3310 may be welded, laminated, thermo-bonded or adhered (e.g. using adhesives) to the conduit 3320. In yet other forms, the pad 3310 may be over-moulded or co-moulded with the conduit 3320. These types of attachment may facilitate a permanent attachment.

However, in forms of the present technology which are shown in FIGS. 9A to 9E, the pad 3310 may be removably attached to the conduit 3320. Arrangements which facilitate such removable attachment will also be discussed further below in more detail.

### 4.3.3.2.1 Pad Materials

In some forms, the pad 3310 may comprise at least one layer of suitable material(s) as known to one skilled in the art. For example, the at least one layer may comprise one or more of a foam, thermoplastic material, a textile material, and a composite material.

In some forms, the pad 3310 may be relatively soft and flexible, e.g. the pad 3310 is made from a relatively soft material having a relatively low modulus of elasticity. An advantage of the pad 3310 being made from a flexible material(s) is that the pad 3310 can move with a non-rigid, i.e. flexible, conduit 3320 to which it is attached. This may also allow the pad 3310 to contour to the shape of the conduit 3320 when attached thereto. In addition, where the pad 3310 is soft, this may make the patient interface 3000 more comfortable for the patient.

In some forms, the pad 3310 comprises layer 3312. Layer 3312 is made from a soft or cushion material, such as a fiber fill and/or a foam material, e.g. polyurethane foam, an Ethylene-vinyl acetate (EVA) foam or a silicone foam.

In some forms, the foam material is a relatively low-density foam material, e.g. it has a density of about 20 to about 30 kg/m³.

In some forms, layer 3312 is made from a material that is resistant to hydrolysis. In some forms, layer 3312 is made from a microbial resistant material. For example, layer 3312 is made from an ether type polyurethane foam.

In some forms, layer 3312 may be configured to absorb and hold moisture such as sweat.

Layer 3312 may be relatively thick e.g. compared to the thickness of at least one of the other layers of the pad(s) 3310. For example, layer 3312 may have a thickness of about 1.5 mm to about 6 mm, preferably about 1.5 mm to about 2 mm.

As illustrated in FIGS. 7A, 10C, 11C, 12C, and FIG. 14C, in some forms, the pad(s) 3310 comprises a plurality of layers, i.e. the pad(s) have a multi-layer structure.

In some forms, the pad(s) 3310 comprises at least one other layer 3314 provided on top of layer 3312. As illustrated, in some forms, layer 3314 is attached to an outer surface of layer 3314, e.g. using polyurethane hot melt dot glue lamination or polyurethane reactive glue. However, layer 3314 may be attached to an outer surface of layer 3312 using one or more other suitable attachment methods known to one skilled in the art. For example, thermoplastic adhesive film, nets and webs for inter-layer attachment or bonding may be used.

In some forms, layer 3314 is relatively soft, e.g. compared to the material from which the conduits 3320 is/are formed. For example, layer 3314 may comprise a soft material, e.g. an eyelet mesh material or textile material. This may help increase patient comfort and/or improve the breathability when wearing the patient interface 3000. Layer 3314 may form an outer layer 3310B of the pad 3310, which may form the patient facing surface of the pad 3310. Alternatively or in addition, the pad may comprise a 3D spacer fabric, e.g. a monofilament spacer fabric or a multifilament spacer fabric. For instance, the layer 3314 may comprise the 3D spacer fabric together with the eyelet mesh material, thereby increasing patient comfort and improving the breathability of the layer 3314.

In some forms, layer 3314 is substantially breathable. For example, layer 3314 may comprise a layer of permeable material, e.g. an eyelet mesh material. Layer 3314 may be relatively permeable compared to at least one other layer of the pad(s) 3310, e.g. layer 3312.

The pad(s) 3310 may be configured to improve moisture control e.g. at least one layer of the pad(s) 3310 may be hydrophilic or hydrophobic. For example, at least one of the layers is hydrophilic and at least one of other layers is hydrophobic. This may improve moisture control and therefore reduce the rate of or eliminate, hydrolysis.

The permeable nature of layer 3314 may allow for improved moisture control in some forms. For example, layer 3314 may facilitate drying out of moisture that collects in layer 3314. This may also improve temperature control and can help cool the patient's head, which can improve patient comfort.

In some forms, the pad(s) 3310 may comprise anti-bacterial properties. For example, layer 3314 may include one or more regions with anti-bacterial properties, e.g. a silver nanofiber. This may limit or prevent the build-up of bacteria in the pad(s) 3310 which may cause odour and even cause infection. This may also improve the patient's comfort and hygiene when using the patient interface 3000 of the present technology.

In some forms, the positioning and stabilising structure 3300 may comprise a substrate layer 3316. For example, the substrate layer 3316 may be provided by a layer of silicone material.

The substrate layer 3316 may be provided to a distal side of the pad 3310 relatively far from the patient's face in use, such that the substrate layer 3316 is positioned between the outer surface 3328 of the conduit 3320 and the conduit facing surface 3310A of the pad 3310. For example, the substrate layer 3316 may be provided closer to the conduit 3320 than layer 3312. For example, in other forms where there are no other layers inferior to layer 3312, the substrate layer 3316 may be applied to surface of layer 3312 facing towards the conduit 3320 in use. Therefore, the substrate layer 3316 may be applied to a surface of the layer of the pad 3310 furthest from the patient in use.

### 4.3.3.2.2 Partially Cured Substrate Layer

In some forms, the substrate layer 3316 may facilitate attachment of the pad 3310, e.g. layer 3312, to a conduit 3320. For example, when attaching the pad 3310 to the conduit 3320, the substrate layer 3316 is provided in a partially cured form (e.g. semi-cured silicone) to the layer of the pad 3310 closest to the conduit and/or to the outer surface 3328 of the conduit 3310. The pad 3310 may then be positioned on the outer surface 3328 such that when the substrate layer 3316 cures, the pad 3310 is attached to the conduit 3320. These aspects of the present technology will be discussed in more detail below.

In some forms, as shown in FIGS. 7A, 10C, 11C, 12C, and FIG. 14C, the pad 3310 may also comprise an intermediate layer 3318 positioned between the substrate layer 3316 and layer 3312. For example, the intermediate layer 3318 may be attached to an inner surface of layer 3312, e.g. using polyurethane hot melt dot glue lamination. However, the intermediate layer 3318 may be attached to an inner surface of the layer 3312 using one or more other suitable attachment methods known to one skilled in the art. In some forms, the intermediate layer 3318 may be attached to an outer surface of substrate layer 3316.

The intermediate layer 3318 may comprise a material which is relatively less permeable compared to at least one other layer of the pad 3310, e.g. layer 3312 and/or layer 3314.

The intermediate layer 3318 may be configured to limit or prevent the substrate layer 3316 and/or adhesives (used to join other layers of the pad(s) 3310) from flowing into at least one other layer of the pad 3310, particularly layer 3312 and/or layer 3314, and setting or curing there. This may ensure that the pad 3310 retains a soft feel or touch and is less likely to become uncomfortable for a patient to wear caused by substrate and/or adhesive which may have leaked into other layers of the pad 3310, and cured or set there.

Furthermore, this may improve attachment of the pad 3310 to the conduit 3320. For instance, more of the semi-cured silicone is retained between the intermediate layer 3318 and the conduit 3320, i.e. less of the semi-cured silicone is absorbed into layer 3312 and/or layer 3314.

In some forms, the pad 3310 comprises at least another intermediate layer 3311 positioned between the substrate layer 3316 and layer 3312. For example, layer 3311 may comprise an adhesive film, e.g. a thermoplastic polyurethane (TPU) adhesive film.

In some forms, layer 3311 may be provided between layer 3318 and substrate layer 3316. For example, layer 3311 is attached to an inner surface of the layer 3318, e.g. using polyurethane hot melt dot glue lamination. However, one or more other suitable attachment methods known to one skilled in the art may be used to attach layer 3311 to layer 3318.

In some forms, the substrate layer 3316 may be applied to an inner surface of layer 3311.

Referring to FIG. 7B, in some forms, the pad(s) 3310 may comprise a first sub-layer 3311a and a second sub-layer 3311b. For instance, the first sub-layer 3331a may be equivalent to the intermediate layer 3318 as is discussed above, while the second sub-layer 3331b may be another material.

The first sub-layer 3331a and the second sub-layer 3331b may be formed from material to provide desirable functions for the pad 3310. For instance, the second sub-layer 3311b may be less permeable than the first sub-layer 3311a. This could be beneficial as it may limit or prevent the substrate layer 3316 and/or other adhesives, from flowing into the first sub-layer 3311a and into another layer of the pad 3310.

In some forms, the first sub-layer 3311a may be breathable. This may work together with layer 3312 to improve moisture and temperature control of the pad 3310.

In some forms, the second sub-layer 3311b may limit or prevent the substrate layer 3316 and/or other adhesives, from flowing into another layer of the pad 3310. For example, the second sub-layer 3311b may be substantially impermeable. Therefore, it can limit or substantially prevent a material such as semi-cured silicone or an adhesive penetrating into the first sub-layer 3311a and into another layer of the pad 3310. This may improve attachment between the pad 3310 and the conduit 3320. In addition, it may stop the adhesive of semi-cured silicone from being positioned in a location in which it would come into contact with a patient's skin; this could improve the comfort of the pad 3310 and therefore the patient interface 3000 in use.

In some forms, the semi-cured silicone may be applied to the conduit 3320. The pad 3310 comprising at least one of the layers described herein may be positioned on (e.g. pressed onto) the semi-cured silicone. In other forms, the semi-cured silicone may be applied to the pad 3310 comprising at least one of the layers described herein, and then positioned on the conduit 3320. In yet other forms, the semi-cured silicone may be applied to the pad 3310 and the conduits 3320, and then the two components may be pressed together. Once the semi-cured silicone cures it will attach the pad(s) 3310 to the conduits 3320. In some forms, the conduit 3320 and pad 3310 may be held together, e.g. proximate each other in a pressed configuration, until the semi-cured silicone cures.

The substrate layer 3316 may be relatively thin compared to the thickness of at least one other layer of the pad 3310. For example, the semi-cured silicone may be applied at a thickness of about 25 μm to about 50 μm.

In some forms, the substrate layer 3316 may be substantially soft and substantially flexible once it cures. In some forms, the substrate layer 3316 may be semi-flexible and semi-rigid after it has cured. This allows the substrate layer 3316 to be bendable and so reduces any impact on the ability of the conduits 3320 to bend in use.

In other forms, the substrate layer 3316 may be at least one of substantially hard and substantially rigid once it has cured.

In certain exemplary forms, the substrate layer 3316 may have:
an elongation of about 130% to about 400%;
a Shore A hardness of about 30 to about 80.

In some forms, the substrate layer 3316 may be substantially transparent or translucent. This may be beneficial as it may permit light to pass through a substantially transparent or translucent conduit 3320, e.g. a silicone conduit, and the substantially transparent or translucent substrate layer 3316 so that the patient can observe from the outside of the conduit 3320 for any dirt that may stick or grime that may build up on the inner surface 3326 of the conduit 3320. Therefore, the substantially transparent or translucent conduit 3320 and the substantially transparent or translucent substrate layer 3316 may together provide a window through which the patient may view the inside of the conduit 3320.

In some forms, the substrate layer 3316 may facilitate attaching the pad 3310 to the conduit 3320. For instance, having the substrate layer 3316 attached to the layer of the pad 3310 closest to the conduit (e.g. layer 3312) before attaching the pad 3310 to the conduit 3320, may allow the properties of the materials of substrate layer 3316 and the conduit 3320 to be selected and a suitable adhesive selected for those materials. In addition, in forms where the substrate layer 3316 is applied in a partially cured form, the subsequent curing of the substrate layer 3316 may facilitate attachment to the conduits 3320. Again, the adhesive may be chosen to facilitate the attachment.

The attachment of materials, e.g. textile materials, to silicone can be difficult and/or expensive. This makes manufacturing positioning and stabilising structures 3300 which comprise silicone conduits 3320 with pad(s) 3310 attached thereto relatively complicated and/or costly. This is particularly relevant where the pad and conduit are manufactured from materials that do not readily bond to the same adhesives. For example, the material from which the layer of the pad closest to the conduit is made and the material from which an outer surface of the conduit is made may have different levels of adhesiveness to a certain adhesive, i.e. one material may form a stronger bond to the adhesive compared to the other material.

The present technology provides an improved way to manufacture the positioning and stabilising structure 3300 which limits or prevents the problems involved with adhesion of a pad 3310 to a silicone conduit 3320. The use of semi-cured silicone may facilitate the attachment between the pad 3310 and the silicone conduit 3320, and therefore improve the manufacturability of the positioning and stabilising structure 3300, by removing the need for or avoiding the use of liquid adhesives to attach the pad 3310 and the silicone conduit 3320 together. This is advantageous as liquid adhesives may bleed-through or flow into at least one layer of the pad 3310 and set or cure there. Therefore, using semi-cured silicone to attach the pad 3310 and the silicone conduit 3320 may be less messy and easier to handle than liquid adhesives.

### 4.3.3.2.3 Removable Pad

In some forms, the positioning and stabilising structure 3300 may comprise a headgear member. The headgear member may comprise a plurality of headgear members. The plurality of headgear members may be connected to each other in use.

As shown in FIGS. 9A to 9E, the headgear member may comprise a conduit 3320 configured to be in fluid communication with the interior of the plenum chamber 3200. The conduit 3320 may be similar in many respects to the conduit 3320 described elsewhere herein, and like references refer to like components. However, the conduit 3320 may differ from other forms in that it comprises features that facilitate removable attachment between the pad 3310 and the conduit 3320 which will be described in more detail below.

It is to be appreciated that in other forms which are not shown, the headgear member may comprise a strap, tie or portion of a headgear system which is positioned on the patient's face and/or head to facilitate holding the seal-forming structure 3100 in a sealing position on a patient's face.

As shown in FIGS. 9A to 9E, in some forms, the pad 3310 may be removably attached to the outer surface 3328 of the patient facing side of the conduit 3320, such that the patient facing surface 3310B of the pad 3310 contacts the patient's face and/or head during use.

In some forms, the pad 3310 and the conduit 3320 may be configured to facilitate this removable attachment. In some forms, the pad 3310 and the conduit 3320 may be configured to connect to each other.

For example, as shown in FIGS. 9C and 9D, the pad 3310 may comprise a projecting member 3330 and the conduit 3320 may comprise an aperture 3340. The aperture 3340 may be configured to receive at least a portion of the projecting member 3330 in an arrangement that holds the pad 3310 and the conduit 3320 together.

At least a portion of the projecting member 3330 may be configured to engage with a surface of the conduit 3320 which at least partially defines the aperture 3340.

In some forms, the projecting member 3330 may include a first projecting member portion 3332 and a second projecting member portion 3334. For example, as shown in FIG. 9D, when viewed in cross-section from a direction which is parallel to a longitudinal extent of the projecting member 3330 (i.e. a direction extending into the page of FIG. 9D), the first projecting member portion 3332 may be narrower than the second projecting member portion 3334. For example, as illustrated in FIGS. 9C and 9D, the projecting member 3330 may have a cross-sectional shape in the form of a dove-tail, or other form of tongue-and-groove connection.

As illustrated, in some forms, the projecting member 3330 may be formed on the conduit facing surface 3310A of the pad 3310. Therefore, the first projecting member portion 3332 may be closer to the conduit facing surface 3310A of the pad 3310 than the second projecting member portion 3334. That is, the second projecting member portion 3334 may form a head section, and the first projecting member portion 3332 may form a stem section, and the head section is located further away from the outer surface 3310B of the pad 3310 than the stem section.

The shape of the aperture 3340 may be complementary to the shape of the projecting member 3330. In some forms, the aperture 3340 may include a first gap 3342 and a second gap 3344, wherein the first gap 3342 is connected to the second gap 3344 which provides an opening 3345 through which the projecting member 3330 can be inserted into the aperture 3340. As shown in FIG. 9D, when viewed in cross-section from a direction which is parallel to the direction extending along the longitudinal length of the aperture 3340 (i.e. a direction extending into the page of FIG. 9D), the second gap 3344 may be narrower than the first gap 3342. For example, the aperture 3340 may have a cross-sectional shape in the form of a dove-tail complementary to the shape of the projecting member 3330, or other form of tongue-and-groove connection.

As illustrated, in some forms, the aperture 3340 may be formed in an outer surface of the headgear member, e.g. the outer surface 3328 of the conduit 3320 facing towards the patient in use. Therefore, the first gap 3342 may be radially further from the central longitudinal axis of the conduit 3320 than the second gap 3344.

In some forms, when attaching the pad 3310 to the conduit 3320, the second projecting member portion 3334 can be inserted into the first gap 3332 via the opening 3345 and through the second gap 3344 such that a surface of the second projecting member portion 3334 (e.g. an underside of the second projecting member portion 3334 as shown in FIG. 9D) engages with a surface of the first projecting member portion 3332 (i.e. a distal surface of the first projecting member portion 3332 as shown in FIG. 9D).

A first part of the conduit 3320 proximate to the opening 3345 may be deformable or flexible relative to a second part of the conduit 3320 adjacent to or surrounding the first part. This may facilitate insertion and removal of the projecting member 3330 into/from the aperture 3340. For example, flaps 3370 may be formed on the outer surface 3328 of the conduit 3320. The flaps 3370 may be thinner than portions of the conduit 3320 adjacent to or surrounding the flaps 3370 to allow this deformation or flexing. However, the flaps 3370 may be configured to be sufficiently stiff or rigid to limit or prevent deformation or flexing that allows the projecting member 3330 to slip out of or dislodge from the aperture 3340 during normal use by the patient such as when moving the patient interface 3000 and components thereof when donning or doffing the patient interface 3000, or when sleeping, for example. Therefore, the flaps 3370 may be stiff or rigid enough to resist forces which are below a certain threshold, e.g forces during normal use, but the flaps 3370 should be flexible or deformable enough to allow deformation or flexing when the certain threshold is exceeded, e.g. forces generated by the patient when pulling the pad 3310 from the conduit 3320.

In other forms, which are not shown, the headgear member, e.g. conduit 3320, may comprise the projecting member 3330, as described above, and the pad 3310 may comprise the aperture 3340, as described above. That is, the ‘male’ and ‘female’ parts of the connection between the conduit and pad may be swapped.

As illustrated in FIGS. 9A to 9E, in some forms, the pad 3310 may comprise a padding portion 3350 and a connector portion 3360. In some forms, the padding portion 3360 may have a similar or the same structure as the pad 3310 described elsewhere herein.

In some forms, the connector portion 3360 may be formed separately and attached to the padding portion 3350. The connector portion 3360 may be permanently attached to the padding portion 3350, for example to a surface of the padding portion 3350 facing away from the patient, and towards the conduit, in use. For example, the connector portion 3360 may be over-moulded to the padding portion 3350. In other forms not shown, the connector portion 3360 may be structured to be removably attached to the padding portion 3350, for example using a hook and loop arrangement (e.g. Velcro™).

As illustrated, in some forms, the connector portion 3360 comprises the projecting member 3330. The connector portion 3360 may comprise a base member 3362. The projecting member 3330 may extend from an inner surface 3362a of the base member 3362 in a direction which extends away from the padding portion 3350. The projecting member 3330 may be substantially perpendicular to a plane defined by the base member 3362. An outer surface 3362b of the base member 3362 may be attached to an inner conduit facing surface 3352 of the padding portion 3350.

The base member 3362 may be thinner than the padding portion 3350, as shown. As shown in FIG. 9D, a width of the base member 3362 between lateral edges may be less than a width of the padding portion between its lateral edges. Therefore, the padding portion 3350 may cover the connector portion 3360.

As described above, in certain other forms which are not shown, the connector portion 3360 may be formed with the aperture 3340 and the protruding member 3330, as described above, may be formed on the outer surface 3328 of the conduit 3320.

The connector portion 3360 may be made from a thermoplastic elastomer (TPE), silicone or from any other suitable material or combination of materials. For example, the connector portion 3360 may be formed by a layer of silicone material. The layer of silicone material may be pre-formed, e.g. injection-moulded, and then attached to the padding portion 3350.

In some forms, as shown in FIGS. 9C and 9D, when viewed in cross-section from a direction which is parallel to the direction along the longitudinal axis of the conduit 3320 (i.e. a direction extending into the page of FIGS. 9D), the first outer surface region 3328a of the conduit 3320 may be substantially flat. In other words, the outer surface 3328 may be substantially level and smooth in this region of the conduit 3320, and may not have curves in the outer surface 3328 that faces towards the patient's face and/or head in use. This arrangement may improve patient comfort. In this example, the first outer surface region 3328a is located on the patient facing side 3302 of the conduit 3320, therefore, the patient facing side 3302 may be substantially flat.

In some forms, as shown in FIG. 9E, as well as in FIGS. 10A, 11A, and 12A, the first outer surface region 3328a of the conduit may be convex-shaped. That is, the outer surface 3328 is curved or rounded in this region of the conduit 3320. The first outer surface region 3328a may be located on the patient facing side 3302 of the conduit 3320, therefore, the patient facing side 3302, or parts thereof, may be substantially curved or rounded. This arrangement may help position the lateral edges 3310C of the pad 3310 further away from the patient's face and/or head and so that the lateral edges 3310C face in a direction extending away from the patient's face and/or head. This may help improve patient comfort because the edges may cause discomfort or facial marking if they come into contact with the face. In this example, the first outer surface region 3328a is located on the patient facing side 3302 of the conduit 3320, therefore, the patient facing side 3302 is substantially convex-shaped.

In some forms, the second outer surface region 3328b may be convex-shaped. That is, this surface is rounded or curved. Therefore, the non-patient facing side 3302 of the conduit 3320 is substantially curved.

In other forms which are not shown, the conduit and the regions described above may have other suitable shapes.

In some forms, as shown in FIGS. 9C to 9E, a joint region 3329 between the first outer surface region 3328a and the second outer surface region 3328b may be convex-shaped. The joint region 3329 is a region where these two regions of the outer surface 3328 connect, and the joint region 3329 may be curved or rounded. This may help avoid sharp corners, edges or joints which can contact the patient's head and/or face in use. This may improve patient comfort and therapy compliance.

As shown in FIG. 9C, in some forms, the projecting member 3330 extends along at least a portion of a length of the inner surface of the pad 3310. The projecting member 3330 may extend along at least a substantial portion of the length of the pad 3310, e.g. along the entire length. This may allow a connector portion 3360 to be formed as an extrusion during manufacture. This may improve manufacturing processes and reduce manufacturing costs. Therefore, in this form the projecting member 3330 may be substantially elongate because a length of the projecting member 3330 is more than the height of the projecting member 3330 (i.e. the distance between the inner surface 3362a of the base member 3362 and an outer-most surface of the projecting member 3330), and more than the width of the projecting member 3330.

Although not illustrated in the figures, in some forms, the aperture 3340 may be provided by a groove which extends along a length of an outer surface of the headgear member, e.g. the outer surface 3328 of the conduit 3320. The groove may be formed in the outer surface 3328 of the patient facing side of the conduit 3320. The groove may also be generally referred to as a “slot” or a “channel”.

The groove may extend along at least a substantial portion of the length of the conduit 3320, e.g. along the entire length. This may allow a conduit 3320 to be formed as an extrusion during manufacture. This may improve manufacturing processes and reduce manufacturing costs.

In other forms not shown in the figures, the projecting member 3330 may extend along a length of an outer surface of the headgear member, and the aperture 3340 may be provided by a groove extending along at least a portion of a length of the conduit facing surface 3310A of the pad 3310.

In yet other forms not shown in the figures, the projecting member 3330 may comprise a plurality of studs spaced apart along the length of the pad 3310, e.g. along the connector portion 3360, and the aperture 3340 may comprise a plurality of holes formed in an outer surface of the headgear member, wherein the plurality of holes are spaced apart along the length of the headgear member, e.g. along the outer surface 3328 of the conduit 3320.

As shown in FIG. 9E, in some forms, the projecting member 3330 comprises a first projecting member 3336 and a second projecting member 3338. The first projecting member 3336 and the second projecting member 3338 may have the same structure, which may be the same as structure of the projecting member 3330 described above. As shown, in some forms, the first projecting member 3336 may be spaced apart from the second projecting member 3338 along the width of the headgear or conduit facing surface 3310A of the pad 3310.

As shown in FIG. 9E, in some forms, the aperture 3340 comprises a first aperture 3346 and a second aperture 3348. The first aperture 3346 may be configured to receive at least a portion of the first projecting member 3336, and the second aperture 3348 may be configured to receive at least a portion of the second projecting member 3338. The first aperture 3346 may be spaced apart from the second aperture 3348 along a perimeter of a patient facing surface of the headgear member, e.g. the outer surface 3328 of the conduit 3320.

Compared to forms in which there is one projecting member and complementary aperture, this arrangement may improve and strengthen the connection between the pad 3310 and the conduit 3320 because two connection points are provided. It is to be appreciated that, in other forms not shown, the conduit 3320 may comprise more than two apertures 3340 and the pad 3310 may comprise more than two projecting members 3330. It is also to be appreciated that, in other forms not shown, the conduit 3320 and the pad 3310 may each have at least one projecting member 3330 and at least one aperture 3340. For example, the first projecting member 3336 and the second aperture 3348 may be formed on the pad 3310 and second projecting member 3338 and the first aperture 3346 may be formed on the conduit 3320.

The connector portion 3360 may be flexible to allow flexing or bending such that the connector portion 3360 can conform to the shape, e.g. curvature, of the outer surface 3328 of the conduit 3320 to which the pad 3310 is attached. Therefore, the connector portion 3360 may be made from a material having a relatively low modulus of elasticity, such as a thermoplastic elastomer (TPE) or silicone, or be made in a form that provides flexibility.

In the alterative form described above but which is not shown, where the projecting member 3330 is formed on the headgear member, e.g. the conduit 3320, the first projecting member 3336 may be spaced apart from the second projecting member 3338 along the perimeter of the outer surface of the headgear member, e.g the outer surface 3328 of the conduit 3328, and the first aperture may be spaced apart from the second aperture along the width of the headgear facing surface 3310A of the pad 3310.

The advantages of the removable pad arrangement described above may include one or more of:
Facilitate replacement of pad 3310, for example if the textile layer of the pad 3310 wears out;
Facilitates cleaning and/or drying of the pad 3310 separately from other components of the patient interface 3000;
Allows the conduit 3320 to be used without the pad 3310, if for example the pad is not dry after cleaning or the pad wears out. The arrangement where the aperture 3340 is formed on the outer surface 3328 of the conduit 3320 may be preferred to accommodate this scenario as opposed to the alternative arrangement (not shown) where the projecting member 3330 is formed on the conduit 3320.

### 4.3.3.3 Conduit(s)

As already described, the conduit 3320 is configured to be in fluid communication with the interior of the plenum chamber 3200.

In some forms, an inferior end region 3321 of the conduit 3320 is configured to be in fluid communication with the inlet of the respective laterally projecting connection portion 3212. For example, the conduit 3320 comprises an opening (not shown in the drawings) formed at the inferior end region 3321.

A superior end region 3323 of the conduit 3320 may be configured to connect to, or terminate with, a connection port 3600, which may be positioned superior to the patient's otobasion superior in use. For example, the connection port 3600 may be positioned on top of the patient's head in use.

In forms where multiple conduits 3320 are present, the superior end regions 3323 of each of the pair of conduits 3320 may be connected to each other. The superior end regions 3323 may connect to a connection port 3600 configured to be positioned on a superior region of the patient's head in use.

In some forms, the pair of conduits 3320 may be integrally formed together. However, in other forms not illustrated, the conduits 3320 may be removably or permanently attached to each other. In yet other forms not illustrated, a connector may be used to facilitate the connection between the conduits 3320, e.g. at their superior end regions 3323, and permit the flow of gases from the connection port 3600 into the conduits 3320. In these forms, the connection port 3600 may be formed as part of the connector.

In some forms, the conduits 3320 are integrally formed with, or permanently attached to, the plenum chamber 3200, as illustrated in FIG. 6A. For example, the conduit 3320 may be moulded or co-moulded with the respective laterally projecting connection portion 3212. In other forms, the conduit 3320 may be removably attached to the plenum chamber 3200. For example, the patient interface 3000 may comprise a connector to facilitate removable attachment of the conduit 3320 to the plenum chamber 3200.

As illustrated, the conduit 3320 comprises an outer surface 3328 and an inner surface 3326. The inner surface 3326 defines the flow path for the passage of pressurised air through the conduit 3320.

In some forms, as described in more detail below, the conduit 3320 may be configured such that one or more regions of the outer surface 3328 may be recessed from one or more other adjacent regions of the outer surface 3328. In terms of the approximately cylindrical geometry of the conduit 3320, the outer surface 3328 of a recessed region may be radially closer to the longitudinal axis of the conduit 3320 than the outer surface 3329 of an adjacent, non-recessed region. That is, when viewed in cross-section from a direction parallel to a direction extending along an axis defined by the flow path (i.e. a transverse cross-section), the outer surface of one or more recessed regions may be closer to the longitudinal axis of the conduit 3320 than the outer surface of the one or more other adjacent regions. In some forms the conduit may have a geometry that is oval or ellipse in cross-sectional shape. In which case, there is variation in the distance of regions of the outer surface of the conduit because of the oval shape in cross-section. It should be appreciated that, in such forms, when referring to one part of the outer surface of the conduit being “radially closer” to the longitudinal axis than another part of the outer surface, this relative distance is indicated as a variation in addition to the variation in distances caused by the oval cross-section.

In some forms, the recessed region(s) may be provided by regions of the conduit 3320 which have a smaller wall thickness (i.e. distance between a point on an inner surface 3326 and a point on the adjacent outer surface 3328) when compared to the wall thickness of the other adjacent regions of the conduit 3320. In some forms, the recessed region(s) may be provided by a change in the shape of the outer surface 3328 between the recessed and non-recessed regions, e.g. a stepped region may be provided between recessed and non-recessed regions of the outer surface 3328.

As described above, in some forms, the conduits 3320 are formed at least in part from silicone. For example, at least the portion of each conduit 3320 to which the pad(s) 3310 is/are attached is made from silicone. Therefore, the outer surface 3328 or parts thereof are made from silicone. In preferred forms, the conduits 3320 are made entirely of silicone. However, in other forms, the conduits 3320 may be formed from any other suitable material or combinations of materials known to one skilled in the art.

In yet other forms, the conduits 3320 may comprise at least one layer of material. For example, the conduits 3320 may comprise a plurality of layers. In one form, at least one of the plurality of layers, e.g. an outer-most layer, comprises silicone.

In certain forms of the present technology, the conduit 3320 is substantially flexible and bendable, e.g. non-rigid. An advantage of this is that the conduit 3320 is more comfortable for a patient to lie upon while the patient is sleeping with their head on the side. However, the flexible and bendable conduits 3320 are configured to be sufficiently rigid or stiffened to limit or prevent occlusion and/or collapse of the conduit 3320 which may impact the air flowing through the flow path. For example, the conduit 3320 may be stiffened. In some forms, stiffening of the conduit 3320 may be achieved by providing at least one of: thicker portions of the conduit 3320; portions of the conduit 3320 made from harder or stiffer materials relative to others portions of the conduit 3320; and reinforcing members or support portions formed with or attached to the conduit 3320.

### 4.3.3.4 Pad and Conduit Arrangements

In certain forms of the present technology, an improved positioning and stabilising structure 3300 is provided which limits or prevents sharp or hard edges and/or joints being formed when attaching the pad(s) 3310 to the conduit 3320. These sharp or hard edges and/or joints may cause discomfort to patients during therapy, and may mark a patient's face, which can reduce compliance with therapy.

For instance, foam laminated textile pads may be cut to a desired size using a radio frequency (RF) cutting tool or other suitable tools. One problem with this process is that, when cutting the foam laminated textile pads (i.e. foam layer laminated with a textile layer) which is to be attached to the conduit 3320, sharp edges and joints may be created. These may be uncomfortable for the patient if contact is made with the patient's skin, for example.

The pad(s) 3310 and conduit 3320 of exemplary forms of the present technology may each be configured and arranged to limit or prevent the formation of sharp or hard edges of the pad 3310, joints between edges of the pad 3310, joints between outer surfaces of the conduit 3320, and/or joints between the pad(s) 3310 and conduit 3320.

The pad(s) 3310 and conduit 3320 may also, or alternatively, be configured and arranged to hide the edges of the pad 3310 and joints between edges of the pad 3310, and/or to position these away from surfaces of the patient's head and/or face. That is, the configurations may position the edges and joints of the pad 3310 on the conduit 3320 so that they face in a direction which extends away from the patient's face and/or head, or position the edges and joints of the pad 3310 relative to the patient's head and/or face so that the edges do not contact the patient's head and/or face in use.

The pad(s) 3310 and conduit 3320 may each be configured and arranged to reduce or prevent the adverse effects of sharp or hard edges and/or joints on the patient interface, e.g. to reduce patient marking or other discomfort.

### 4.3.3.4.1 First Arrangement

One way of achieving one or more of the objectives of the present technology is to provide an arrangement in which the pad(s) 3310 sits substantially flush with a portion of the conduit 3320 when attached thereto. The outer surface 3328 of the conduit 3320 may be configured such that, when the pad 3310 is positioned on the first outer surface region 3328a, a joint between the outer surface 3310B of the pad 3310 and the outer surface 3328 in the second outer surface region 3328b is substantially flush.

As mentioned above, in some forms, one or more regions of the outer surface 3328 of the conduit 3320 may be recessed from one or more other adjacent regions of the outer surface 3328. For instance, the first outer surface region 3328a of the conduit 3320 may be recessed from the second outer surface region 3328b. The first outer surface region 3328a may be referred to as a recessed region 3322. The second outer surface region 3328b may be referred to as portion 3324.

As illustrated in FIGS. 10A to 14C, the recessed region 3322 may be defined by at least one connecting wall 3322b and a recessed wall 3322c. For example, the recessed region 3322 may be defined by a first and second connecting wall 3322b and the recessed wall which extends between the first and second connecting walls 3322b. The recessed wall 3322c is recessed from an outer wall 3324a of the conduit 3320 located in the second outer surface region 3328b. That is, the recessed wall 3322c may be located radially more closely to the longitudinal axis of the conduit 3320 relative to the outer wall 3324a. A joint region 3322a may formed between the connecting wall 3322b and the outer wall 3324a. The recessed region may be provided by a step, or may be referred to as a “recess” or “depression” which is formed in the outer surface 3328.

The pad 3310 is shaped and dimensioned to be located in the recessed region 3322 between the first and second connecting walls 3322b, which is configured to limit or prevent the creation of the sharp or hard edges and/or joints as described above. For example, the pad(s) 3310 may be shaped and dimensioned to fit into the recess 3322 so that the outer surface 3310B of the pad(s) 3310 sits substantially flush with the patient facing surface 3328 of the conduit 3320. In some forms, as illustrated in FIGS. 10C, 11C, 12C and FIG. 14C the pad(s) 3310 may comprise a plurality of layers (as described herein).

In the embodiment of FIGS. 10A to 10C, the pad 3310 is positioned, e.g. seated, in the recessed region 3322 and no portion of the pad 3310 is folded, tucked under itself, or positioned under a portion or surface of the conduit 3320. The pad 3310 may be formed so that it has even or flat edges, e.g. by cutting or trimming the layers of the pad 3310 so that the edges can sit flush against connecting wall 3322b.

### 4.3.3.4.2 Second Arrangement

In some forms, the pad 3310 may comprise a first pad region 3313a and a second pad region 3313b. The first and second pad regions 3313a and 3313b may be regions at or proximate the lateral edge of the pad 3310.

In some forms, for example as shown in FIGS. 11A to 11C, the first pad region 3313a may be folded under the second pad region 3313b such that at least one of the edges 3310C, 3310D of the pad 3310 is positioned between the outer surface 3328 of the conduit 3320 and a conduit facing surface of the second pad region 3313b. For instance, in the illustrated example, the lateral edges 3310C are positioned between the outer surface 3328 of the conduit 3320 and a conduit facing surface of the second pad region 3313b. In other words, a portion of the pad 3310, e.g. the first pad region 3313a, may be folded under itself to create a folded region. The folded region may create a substantially smooth edge or a more rounded edge of the pad 3110, as opposed to an edge of the pad 3310 that is cut and not folded. The edge created by the folded region may be positioned to abut or to be proximate the connecting wall 3322b. Therefore, again in this form, the pad is positioned between the first and second connecting walls 3322b.

In some forms, a groove 3322d may be formed in the outer surface 3328 of the conduit 3320. The groove 3322d may extend along at least a portion of a length of the conduit 3320, e.g. along a substantial portion of, or the entire, length of the conduit. In some forms, the groove 3322d comprises a first and second groove 3322d spaced apart along the perimeter of the outer surface 3328 of the conduit 3320.

As shown in FIGS. 11A to 11C, in some forms, the first pad region 3313a (i.e. the folded region) may be positioned in the groove 3322d.

In some forms, as shown in FIGS. 11A to 11C, a portion of the recessed region 3322 may be relatively deeper compared to an adjacent portion 3322e of the recessed region 3322 (i.e. the deeper portion is radially more proximate the longitudinal axis of the conduit 3320 relative to the adjacent portion 3322e). The deeper portion may be provided by the groove 3322d. The groove 3322d may be formed in the recessed wall 3322c. The first and second grooves 3322d may each be located proximate the respective first and second connecting wall 3322b.

### 4.3.3.4.3 Third Arrangement

In some forms, as shown in FIGS. 12A to 12C, an overhang 3322f may be formed in the outer surface 3328 of the conduit 3320. For example, the outer surface 3328 may be configured such that a first outer surface of the conduit 3320 overhangs a second outer surface of the conduit 3320 to create a notch 3322h which extends between the first outer surface and the second outer surface. At least one of the edges 3310C, 3310D of the pad 3310, or at least part thereof, e.g. the lateral edges, is positioned in the notch 3322h. The edge(s) 3310C, 3310D may be positioned under the first outer surface. In the illustrated example, the lateral edges 3310C are positioned in the notch 3322h. The first pad region 3313a can be inserted into the notch 3322h defined by the overhang 3322f.

In some forms, the connecting wall 3322b may be structured to create the overhang 3322f. For example, as illustrated in FIGS. 12B and 12C, the overhang 3322f may be provided by a radially outer section 3322g of the connecting wall 3322b which protrudes into the recess 3322. That is, the radially outer section 3322g is a section which is positioned further from the central longitudinal axis of the conduit 3320 than an adjacent, inferior section. In yet other exemplary forms which are not shown, the connecting wall 3322b may be curved to create the overhang 3322f, at least in part. The first outer surface of the conduit which overhangs the second outer surface to create the notch 3322h may be provided by an underside of the radially outer section 3322g, and the second outer surface may be provided by a portion of the recessed wall 3322c.

In some forms, as shown in FIGS. 12A to 12C, the groove 3322d is configured on the outer surface 3328 to form the overhang 3322f and the notch 3322h. The first and second grooves 3322d may each be formed in the respective first and second connecting wall 3322b

As shown in FIGS. 12A and 12B, in some forms, the first pad region 3313a may be tucked into the groove 3322d. This may compress at least parts of the first pad region 3313a to fit into the gap 3322h which has a height (i.e. distance between the recessed wall 3322c and the underside of the radially outer section 3322g) which is smaller than a height of the connecting wall 3322b (or the depth of the recessed portion 3322). In other forms, the first pad region 3313a may tuck into the groove 3322d without needing to be compressed.

As previously described, in some forms, each pad 3310 may comprise a plurality of layers. In some forms, at least one of the layers of the pad(s) 3310 may have greater dimensions, e.g. width, compared to the dimensions, e.g. width, of at least one of the other layers of the pad(s) 3310. In some forms, the wider layer(s) can be tucked under the overhang 3322f, as illustrated in FIG. 12C. For example, layer 3312 and layer 3314 may have less width relative to the width of layer 3316 and layer 3318 which tuck under the overhang 3322f. This may involve forming the pad(s), e.g. by cutting at least one layer of the pad(s) 3310, e.g. layer 3312 and layer 3314, to have different dimensions compared to the other layer(s) of the pad(s) 3310.

### 4.3.3.4.4 Fourth Arrangement

As shown in FIGS. 13A and 13B, in some forms, the recessed region 3322 may be positioned adjacent a regions of the outer surface 3328 of the conduit 3320 which have a higher degree of curvature than other regions of the outer surface of the conduit. In preferred forms, the recessed region 3322 may be located between two regions of the outer surface 3328 which have such a higher degree of curvature. That is, these regions may be located on either side of the recessed region 3322 and may have highly curved or rounded surfaces. This configuration may help provide a smoother, less sharp joint between respective outer surfaces of the conduit 3320, which may improve patient comfort, and may improve aesthetic appeal of the conduit 3320.

The recessed region 3322 may help position the lateral edges 3310C of the pad 3310 further away from the patient's face and/or head. The recessed region 3322 may be shaped so that the lateral edges 3310C may be positioned to face in a direction extending away from the patient's face and/or head, e.g. lateral edge regions of the pad 3310 may curve away from the patient's face. This may also help hide any sharp, uneven, rough edges, joints and/or seams on the pad 3310. This arrangement may help improve patient comfort.

In some forms, portions (e.g. layers) of the pad 3310 may be laminated or welded together, or attached together otherwise, and in this attachment may form a substantially sharp joint which may be located on the edge 3310C, 3310D. Therefore, the arrangements of FIGS. 13A and 13B are configured to help hide this sharp joint, for example, by bending lateral edge regions of the pad 3310 away from the patient's face, and/or by positioning the pad 3310 in the recessed region 3322. In one form, these portions of the pad 3310 may be joined together at a point on the edge 3310C, 3310D which is positioned nearer to the conduit 3320 when the pad 3310 is attached to the conduit 3320. This may help keep any sharp joints away from contact with the patient's skin.

In some forms, the recessed region 3322 may facilitate placement or positioning of the pad 3310 on the conduit 3320, for example during manufacture. That is, the pad 3310 may nest in the conduit 3320, and the recessed region 3322 may be configured to facilitate such nesting. The recessed region 3322 may create a negative space on the outer surface 3328 of the conduit 3320 into which the pad 3310 may be positioned.

In some forms, the connecting wall 3322b and the second outer surface region 3328b are shaped such that a joint region between the connecting wall 3322b and a surface of the second outer surface region 3328b is convex-shaped with a relatively high degree of curvature compared to other regions of the outer surface of the conduit.

In some forms, as shown in FIG. 13A, at least a portion of the first outer surface region 3328a is substantially flat, e.g. the patient facing side of the conduit may be substantially flat, as shown.

In some forms, as shown in FIG. 13B, at least a portion of the first outer surface region 3328a is convex-shaped, for example a substantial portion may be convex-shaped. That is, at least a portion of the first outer surface region 3328a is curved or rounded. In other forms, at least a portion of the first outer surface region 3328a is concave-shaped. The first outer surface region 3328a may have a first portion 3328c and an adjacent second portion 3328d. FIG. 13B shows a form of the technology where the first portion 3328c may be convex-shaped and the second portion 3328d may be concave-shaped. As shown, the second portion 3328d may be located between the second outer surface region 3328b and the first portion 3328c. The second portion 3328d may comprise portions of the first outer surface region 3328a which are located on either side of the first portion 3328c. Therefore, the recessed region 3322 may have regions which are convex-shaped and concave-shaped.

In some forms, at least one of the edges 3110C, 3110D of the pad 3110 may be positioned on the second portion 3328d. For example, as shown in FIG. 13B, at least a portion of the lateral edges 3110C are positioned on the concave-shaped second portion 3328d.

In some forms, as shown in FIG. 13B, the connecting wall 3322b and the first outer surface region 3328a are shaped such that a joint region between the connecting wall 3322b and the first outer surface region 3328a is concave-shaped. In some forms, the joint region may form part of the concave-shaped second portion 3328d. In some forms, at least a portion of the lateral edges 3110C may be positioned on the joint region. In some forms, at least a portion of the lateral edges 3110C may be positioned on the connecting wall 3322b, as illustrated.

In some forms, the recessed region 3322 may be configured to contour the shape of the pad 3310. For example, as illustrated in FIG. 13, the first outer surface region 3328a, the joint region and the connecting wall 3322b may be shaped to contour the shape of a portion of the pad 3310.

In some forms, the concave-shaped second portion 3328d, the concave-shaped joint region described above, and/or the shape of the connecting wall 3322b, may help limit or prevent the formation of sharp, rough, hard, uneven edges, surfaces and/or joints on the outer surface 3328 of the conduit 3320.

These arrangements may help limit or prevent the edges 3110C, 3110D and joints of the pad 3110 from contacting surfaces of the patient's face and/or head. This is because the shape of the first outer surface region 3328a, e.g. convex-shaped, allows the edges 3110C, 3110D and joints of the pad 3310 to be positioned so that they face in a direction which extends away from the patient's face and/or head.

The arrangements of the pad(s) 3310 and recessed region 3322, and methods of manufacture, as described herein may limit or prevent the creation of sharp, hard, uneven edges and/or joints between edges or surfaces.

This may improve patient comfort when using the patient interface 3000. This may also improve the manufacturing process without substantially increasing manufacturing times or costs.

### 4.3.3.4.5 Fifth Arrangement

Another arrangement to achieve one or more of the objectives of the present technology is to structure the conduit 3320 to ensure that a joint region between the outer surface 3310B and/or the edge(s) 3310C, 3310D of the pad(s) 3310 and the outer surface 3320 of the conduit 3320 is positioned in use away from the patient's face and/or head. The pad 3110 may not abut or contact the conduit 3320 at the joint region, but they may be positioned proximate to each other.

As shown in FIGS. 14A to 14C, in some forms, the pad 3110 may be positioned to lie over the second outer surface (i.e. non-recessed) region 3328b. In these forms, the second outer surface region 3328b is located on the patient facing side 3302. Therefore, this embodiment differs from the other embodiments described above in that at least a substantial portion of the pad 3310, e.g. the entire pad 3310, may be positioned on the second outer surface (non-recessed) region 3328b. That is, in some forms, the pad 3310 is not provided to the first outer surface region 3328a at all. In other forms, a portion of the pad 3310 may be positioned on the first outer surface region 3328a, however, at least a substantial portion of the pad 3310, e.g. the entire pad 3310, is provided to the second outer surface region 3328b.

In some forms, as shown in FIGS. 14A to 14C, the conduit 3320 may be configured so that a portion of the pad(s) 3310 is positioned to lie over the joint region 3322a. The conduit 3320 may be provided with the recessed region 3322, however, as described above, the pad 3110 may be positioned differently on the conduit 3320.

The portion 3324 may be considered to protrude in a direction (e.g. a radial direction) facing away from the longitudinal axis defined by the conduit 3320. Therefore, the portion 3324 may be referred to as a “protruding portion”, wherein the outer wall 3324a provided on portion 3324 may protrude relative to the recessed wall 3322b. Put differently, the outer wall 3324a may be raised relative to the recessed wall 3322c.

In some forms, the portion 3324 may help position surrounding portions of the conduit 3320, e.g. outer surface 3328, away from the patient's face and/or head in use. This is because portion 3324 protrudes relative to the surrounding portions of the outer surface 3328, and portion 3324 is proximal the patient's head and/or face, in use, relative to the surrounding portions, such that the surrounding portions are positioned further away from surfaces of the patient's head and/or face than the protruding portion is, in use.

In some forms, as shown in FIGS. 14A to 14C, the connecting wall 3322b may slope between the recessed wall 3322c and the outer wall 3324a.

However, in other forms, for example as illustrated in FIGS. 13A and 13B, a smoother, more rounded joint region 3322a may be provided, and/or a smoother, more rounded joint region between the recessed wall 3322c and the connecting wall 3322b may be provided. For instance, as described above, the outer surface 3328 in these regions of the conduit 3320 may be substantially curved or rounded so that the formation of a sharp edge or joint is limited or prevented.

In some forms, the pad 3110 may be positioned to lie over the second outer surface region 3328b such that at least one of the lateral edges 3110C, 3110D of the pad 3110 is positioned on the first outer surface region 3328a. The pad(s) 3310 may be positioned to lie over the joint region 3322a, e.g. to completely cover the connecting wall 3322b. In some forms, the first pad region 3313a lies over the entire connecting wall 3322b, and the second pad region 3313b lies over the outer surface 3324a. In a first form, at least one of the lateral edges 3310C, 3310D of the pad 3110 may be positioned against or abuts a part of the outer surface 3328 located in the first outer surface region 3328a, e.g. on the recessed wall 3322c. In a second form, at least one of the lateral edges 3310C. 3310D may be positioned on the outer surface 3328 located in the first outer surface region 3328a, e.g. on the recessed wall 3322c. These forms may help hide the edge(s) 3110C. 3110D and joints of the pad 3110. The first form may help provide a smoother joint region between the edge of the pad 3110, e.g. lateral edge 3110C, and the outer surface 3328 of the conduit 3320 when compared to a joint region created if the edge 3110C were to extend over part of the outer surface 3328 or extend partially over the connecting wall 3322b. However, in other forms (not shown), these other arrangements may be provided, and the edge 3110C may still be hidden or positioned away from the patient's head and/or face. Therefore, in other forms (not shown), the pad 3110 may be positioned on the second outer surface region 3328b and not on the first outer surface region 3328a, or to a lesser extent on the first outer surface region 3328a. In this arrangement, the pad 3110 may be positioned to lie over the second outer surface region 3328b such that at least one of the edges 3110C. 3110D is positioned on the connecting wall 3322b.

In some forms, the conduits 3320 may be configured to limit or prevent turbulence and noise being created in the conduits 3320. For example, the inner surface 3326 of the conduit 3320 which defines the flow path of the conduit 3320 may not have a recessed region which may be created when forming the conduits 3320. The presence of a recessed region on the inside of the conduit 3320 may affect gas flow in the conduit 3320. This may be disruptive to patients and lead to non-compliance with therapy. Therefore, as shown in the figures, the inner surface 3326 may be substantially smooth, and joint regions between surfaces or regions of the inner surface 3326 may be curved or rounded to improve the flow of air through the flow path of the conduit 3320.

### 4.3.4 Connection Port

Connection port 3600 allows for connection of the patient interface 3000 to the air circuit (not shown). The connection port 3600 receives the flow of breathable gas from the air circuit.

In some forms, the positioning and stabilising structure may comprise one or more conduits 3320, for example as part of a headgear conduit system. The connection port 3600 may be provided to, or formed in, the one or more conduits 3320 so that the connection port 3600 allows the flow of breathable gas from the air circuit to the one or more conduits 3320. In some forms, the patient interface 3000 may be configured so that, when worn, the connection port 3600 is positioned superior to the patient's otobasion superior. For example, the connection port 3600 may be positioned on top of the patient's head in use.

In some forms, the patient interface 3000 comprises an air circuit, e.g. an air delivery conduit. The air delivery conduit may be located upstream of the connection port 3600 and may deliver the flow of breathable gas from the air circuit to the connection port 3600.

### 4.3.5 Vent Structure

A patient interface 3000 according to some examples of the present technology comprise a vent structure 3400 configured to allow a flow of gases exhaled by the patient from an interior of the plenum chamber 3200 to ambient. The vent structure 3400 may be configured such that the vent flow rate has a magnitude sufficient to reduce rebreathing of exhaled CO2 by the patient while maintaining the therapeutic pressure in the plenum chamber 3200 in use.

In preferred forms, the vent structure 3400 comprises a plurality of vent holes 3410.

In some forms, the vent structure 3400 may be provided to the plenum chamber 3200, as shown in FIG. 9A.

### 4.3.6 Diffuser

A patient interface 3000 according to some examples of the present technology comprise a diffuser (not shown) to diffuse the flow of gases. The diffuser may be positioned in use to diffuse the flow of gases exiting the vent structure 3400.

### 4.3.7 Decoupling Structure(s)

In one form the patient interface 3000 includes at least one decoupling structure, for example, a swivel or a ball and socket.

### 4.3.8 Forehead Support

In one form, the patient interface 3000 includes a forehead support 3700.

### 4.3.9 Anti-Asphyxia Valve

In one form, the patient interface 3000 includes an anti-asphyxia valve.

### 4.3.10 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 METHOD OF MANUFACTURING POSITIONING AND STABILISING STRUCTURE

Forms of the technology provide a method of manufacturing a positioning and stabilising structure 3300.

In certain forms, the method may comprise the following steps, occurring in any order:
(a) forming at least one pad 3310;
(b) forming at least one conduit 3320; and
(c) attaching the at least one pad 3310 to the at least one conduit 3320.

### 4.4.1 Formation of Pad(s)

In certain forms, step (a) comprises forming the pad(s) 3310 comprising at least one layer.

In certain forms, step (a) comprises forming the pad(s) 3310 comprising a plurality of layers.

In certain forms, step (a) comprises forming the pad(s) 3310 comprising layer 3312 and layer 3316.

In certain forms, step (a) comprises forming the pad(s) 3310 comprising layer 3314.

In certain forms, step (a) comprises providing layer 3314 on top of layer 3312. For example, this step may include laminating the layer 3314 to an outer surface of the layer 3312.

In certain forms, step (a) comprises forming the pad(s) 3310 comprising layer 3318.

In certain forms, step (a) comprises positioning layer 3318 between layer 3312 and layer 3316. For example, this step may include laminating layer 3318 to an inner surface of layer 3312.

In certain forms, step (a) comprises forming the pad(s) 3310 comprising layer 3311.

In certain forms, step (a) comprises providing layer 3311 inferior to layer 3312. For example, this step may include laminating layer 3311 to an inner surface of layer 3318.

In certain forms, step (c) comprises applying layer 3316 inferior to layer 3312. For example, this step may include coating semi-cured silicone onto an inner surface of layer 3311.

In certain forms, step (a) comprises forming the semi-cured silicone.

In certain forms, step (a) comprises forming the pad(s) 3310 to be shaped and dimensioned to fit into the recessed region 3322 formed on the at least one conduit 3320. For example, this step may include cutting the pad(s) 3310, or layers thereof, to have a predetermined shape and dimensions.

In certain forms, step (a) comprises forming the pad(s) 3310 to be shaped and dimensioned to fit into the recessed region 3322 formed on the at least one conduit 3320 and the first pad region 3313a of the pad(s) 3310 is folded and tucked under the second pad region 3313b to form the folded region.

In certain forms, step (a) comprises forming the pad(s) 3310 to be shaped and dimensioned to fit into the recessed region 3322 formed on the at least one conduit 3320 and the first pad region 3313a of the pad(s) 3310 is positioned in the gap 3322h and under the overhang 3322f.

In certain forms, step (a) comprises forming the pad(s) 3310 to be shaped and dimensioned to lie over the joint 3322a formed on the at least one conduit 3320, and an edge 3310C, 3310D of the pad(s) is positioned against, to abut, or proximate to an outer surface 3328 of the conduit 3320. For example, this step may include forming the pad(s) 3310 to be shaped and dimensioned to lie over the joint 3322a formed by the portion 3324 formed on the at least one conduit 3320.

### 4.4.2 Formation of Conduit

In certain forms, step (b) comprises forming at least one conduit 3320 which is formed at least in part from silicone. For example, this step may comprise forming a pair of conduits 3320.

In certain forms, step (b) comprises connecting superior end regions 3323 of the conduits 3320 to each other. For example, this step may comprise integrally forming the conduits 3320 together.

In certain forms, step (b) comprises forming a recessed region 3322 in the conduits 3320, wherein the recess has the joint 3322a.

In certain forms, step (b) comprises forming the recessed region 3322 with the connecting wall 3322b which extends in a substantially perpendicular direction from the recessed wall 3322c of the recess 3322.

In certain forms, step (b) comprises forming the recessed region 3322 with a portion of the recessed wall 3322c that is deeper with respect to an adjacent portion 3322e of the recessed wall 3322c. For example, this step may comprise forming the groove 3322d with a depth which is greater than the depth of the adjacent portion 3322e.

In certain forms, step (b) comprises forming the recessed region 3322 with the overhang 3322f.

In certain forms, step (b) comprises forming the joint 3322a on the conduits 3320. For example, this step may comprise forming the conduits 3320 with the portion 3324, wherein the portion 3324 provides the joint 3322a.

In certain forms, step (b) comprises forming the portion 3324 with an outer wall 3324a and the connecting wall 3322b which extends at a non-perpendicular angle with respect to the axis defined by the flow path of the conduit 3320.

In certain forms, step (b) comprises forming the conduits 3320 not to have a recessed region on an inner surface 3326.

### 4.4.3 Attachment of Pad(s) to Conduits

In certain forms, step (c) comprises attaching layer 3312 to the conduit 3320. For example, this step may include positioning the pad(s) 3310 which comprises semi-cured silicone on a silicone conduit 3320.

In certain forms, step (c) comprises positioning the pad(s) 3310 on the conduit 3320 so that an outer surface 3310B of the pad(s) 3310 is positioned to be flush with an outer surface 3328 of the conduit 3320.

In certain forms, step (c) comprises positioning the pad(s) 3310 on the conduit 3320 so that the pad(s) 3310 is positioned in the recess 3322 and no portion of the pad(s) 3310 is folded and tucked under itself, or positioned under a portion of the conduit 3320.

In certain forms, step (c) comprises positioning the pad(s) 3310 to the conduit 3320 so that the first pad region 3313a of the pad(s) 3310 is folded and tucked under the second pad region 3313b. For example, this step may include positioning the pad(s) 3310 to the conduit 3320 so that the first pad region 3313a, i.e. the folded region is positioned in the groove 3322d.

In certain forms, step (c) comprises positioning the pad(s) 3310 on the conduit 3320 so that at least a portion of the first pad region 3313a of the pad(s) 3310 is positioned under the overhang 3322f.

In certain forms, step (c) comprises positioning the pad(s) 3310 on the conduit 3320 so that at least a portion of the pad(s) 3310 is positioned to lie over the joint 3322a. For example, this step may include positioning the pad(s) 3310 to the conduit 3320 so that the edge(s) 3310C, 3310D is positioned against, proximate to, or to abut the outer surface 3328 of the conduit 3320.

In certain forms, step (c) comprises curing layer 3312. For example, this step may include curing layer 3312 at predetermined conditions. Layer 3312 may be cured at a predetermined temperature, e.g. the semi-cured silicone is cured at a temperature of about 90° C. to about 150° C. Layer 3312 may be cured for a predetermined period of time, e.g. the semi-cured silicone is cured for about 15 minutes to about 60 minutes. Layer 3312 may be cured at a predetermined pressure, e.g. the semi-cured silicone is cured under a pressure of about 0.1 Pa to about 2 Pa.

In certain forms, step (c) comprises curing layer 3312 in a first step and a second step. For example, the first step may include curing layer 3312 at a first set of predetermined conditions, e.g. curing the semi-cured silicone at a temperature of about between 90° C. to about 120° C. for about 15 minutes to about 30 minutes. For example, the second step may include curing layer 3312 at a second set of predetermined conditions, e.g. curing the semi-cured silicone at a temperature of about 120° C. to about 150° C. for about 15 minutes to about 30 minutes, under a pressure of about 0.1 Pa to about 2 Pa.

### 4.5 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 4.5.1 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.6 Air Circuit

An air circuit 4170 in accordance with an aspect of the present technology is a conduit or a tube constructed and arranged to allow, in use, a flow of air to travel between two components such as RPT device 4000 and the patient interface 3000.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 4.7 Humidifier

### 4.7.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.8 GLOSSARY

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜ 0.001 atm). In this specification, unless otherwise stated, pressure is given in units of cmH₂O.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.8.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240

Polycarbonate: a thermoplastic polymer of Bisphenol-A Carbonate.

### 4.8.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.8.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort.

Hyperpnea: An increase in flow to a level higher than normal.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.8.3 Anatomy

### 4.8.3.1 Anatomy of the Face

Ala: the external outer wall or “wing” of each nostril (plural: alar)

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Frankfort horizontal plane: A line extending from the most inferior point of the orbital margin to the left tragion. The tragion is the deepest point in the notch superior to the tragus of the auricle.

Nares (Nostrils): Approximately ellipsoidal apertures forming the entrance to the nasal cavity. The singular form of nares is naris (nostril). The nares are separated by the nasal septum.

Otobasion superior: The highest point of attachment of the auricle to the skin of the face.

Sagittal plane: A vertical plane that passes from anterior (front) to posterior (rear). The midsagittal plane is a sagittal plane that divides the body into right and left halves.

### 4.8.3.2 Anatomy of the Skull

Nasal bones: The nasal bones are two small oblong bones, varying in size and form in different individuals; they are placed side by side at the middle and upper part of the face, and form, by their junction, the “bridge” of the nose.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.8.4 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.8.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 4.8.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 4.8.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

### 4.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.10 REFERENCE SIGNS LIST

1000 Patient
1100 Bed partner
3000 Patient interface
3100 Seal-forming structure
3150 Cushion module
3200 Plenum chamber
3210 Frame portion
3212 Laterally projecting connection portion
3300 Positioning and stabilising structure
3302 Patient facing side
3304 Non-patient facing side
3310 Pad(s)
3310A Inner surface
3310B Outer surface
3310C Lateral edges
3310D End edges
3311 Layer
3311a First sub-layer
3311b Second sub-layer
3312 Layer of cushioning material
3313a First pad region
3313b Second pad region
3314 Layer
3316 Substrate layer
3318 Intermediate layer
3320 Conduit
3321 Inferior end region
3322 Recessed region
3322a Joint
3322b Connecting wall
3322c Recessed wall
3322d Groove
3322e Adjacent portion
3322f Overhang
3322g Radially outer section
3322h Notch
3323 Superior end region
3324 Portion
3324a Outer wall
3325 Connector
3325a Slot
3326 Inner surface
3328 Outer surface
3328a First outer surface region
3328b Second outer surface region
3328c First portion
3328d Second portion
3329 Joint region
3330 Projecting member
3332 First projecting member portion
3334 Second projecting member portion
3336 First projecting member
3338 Second projecting member
3340 Aperture
3342 First gap
3344 Second gap
3345 Opening
3346 First aperture
3348 Second aperture
3350 Padding portion
3352 Inner surface
3360 Connector portion
3362 Base member
3362a Inner surface
3362b Outer surface
3370 Flaps
3400 Vent structure
3500 Strap
3510 Fabric outer layer
3512 Portion
3514 Another Portion
3600 Connection port
4000 RPT device
4010 External housing
4012 Upper portion
4014 Lower Portion
4015 Panel
4016 Chassis
4018 Handle
4020 Pneumatic block
4100
Blower housing
Air filter
4110
4112 Inlet air filter
4114 Outlet air filter
4120 Muffler
4122 Inlet muffler
4124 Outlet muffler
4140
Pressure generator
Blower
4144 Motor

1-64. (canceled)

65. A positioning and stabilising structure configured to hold a seal-forming structure of a patient interface in a sealing position on a patient's face, the positioning and stabilising structure comprising:
a conduit configured to permit delivery of a flow of air at a therapeutic pressure into a plenum chamber of the patient interface through an inlet formed in the plenum chamber, wherein the conduit comprises a patient facing side and a non-patient facing side; and
a pad comprising a conduit facing surface, an opposing patient facing surface, opposing lateral edges and opposing end edges, wherein the conduit facing surface of the pad is attached to an outer surface of the conduit on the patient facing side, wherein the pad comprises a first pad region and a second pad region, wherein the first pad region is folded under the second pad region such that at least one of the edges of the pad is positioned between the outer surface of the conduit and a conduit facing surface of the second pad region, and such that a patient facing surface of the second pad region contacts the patient's head and/or face in use.

66. The positioning and stabilising structure according to claim 65, wherein the outer surface of the conduit comprises a first outer surface region and a second outer surface region, wherein the conduit comprises first and second connecting walls connecting lateral edges of the first outer surface region with respective lateral edges of the second outer surface region, and wherein the pad is positioned on the first outer surface region between the first and second connecting walls.

67. The positioning and stabilising structure according to claim 66, wherein the first outer surface region is located on the patient facing side and the second outer surface region is located on the non-patient facing side.

68. The positioning and stabilising structure according to claim 66, wherein the outer surface of the conduit is configured such that the first outer surface region is recessed from the second outer surface region.

69. The positioning and stabilising structure according to claim 66, wherein the outer surface of the conduit is configured such that, when the pad is positioned on the first outer surface region, a joint region between the patient facing surface of the second pad region and an outer surface of the second outer surface region of the conduit is substantially flush.

70. The positioning and stabilising structure according to claim 65, wherein a groove is formed in the outer surface of the conduit, wherein the groove extends along at least a portion of a length of the conduit, and wherein the first pad region is positioned in the groove.

71. The positioning and stabilising structure according to claim 70, wherein the groove is formed in the first outer surface region.

72. The positioning and stabilising structure according to claim 65, wherein the conduit comprises first and second conduits, wherein the positioning and stabilising structure is configured such that, in use, the first conduit is positioned on one side of the patient's face and the second conduit is positioned on the other side of the patient's face.

73. The positioning and stabilising structure according to claim 65 further comprising at least one headgear strap attached to the conduit.

74. A positioning and stabilising structure configured to hold a seal-forming structure of a patient interface in a sealing position on a patient's face, the positioning and stabilising structure comprising:
a conduit configured to permit delivery of a flow of air at a therapeutic pressure into a plenum chamber of the patient interface through an inlet formed in the plenum chamber, wherein the conduit comprises a patient facing side and a non-patient facing side; and
a pad comprising a conduit facing surface, an opposing patient facing surface, opposing lateral edges and opposing end edges, wherein the conduit facing surface is attached to an outer surface of the conduit on the patient facing side, wherein the patient facing surface of the pad contacts the patient's head and/or face in use,
wherein the outer surface of the conduit is configured such that a first outer surface of the conduit overhangs a second outer surface of the conduit and creates a notch extending between the first outer surface and the second outer surface, and wherein at least one of the edges of the pad is positioned in the notch.

75. The positioning and stabilising structure according to claim 74, wherein the notch extends along at least a portion of a length of the conduit.

76. The positioning and stabilising structure according to claim 74, wherein the outer surface of the conduit comprises a first outer surface region and a second outer surface region, wherein the conduit comprises first and second connecting walls connecting lateral edges of the first outer surface region with respective lateral edges of the second outer surface region, and wherein the pad is positioned on the first outer surface region between the first and second connecting walls.

77. The positioning and stabilising structure according to claim 76, wherein the first outer surface region is located on the patient facing side and the second outer surface region is located on the non-patient facing side.

78. The positioning and stabilising structure according to claim 76, wherein the outer surface of the conduit is configured such that the first outer surface region is recessed from the second outer surface region.

79. The positioning and stabilising structure according to claim 76, wherein the outer surface of the conduit is configured such that, when the pad is positioned on the first outer surface region a joint region between a patient facing surface of the pad and an outer surface of the second outer surface region is substantially flush.

80. The positioning and stabilising structure according to claim 74, wherein the conduit comprises first and second conduits, wherein the positioning and stabilising structure is configured such that, in use, the first conduit is positioned on one side of the patient's face and the second conduit is positioned on the other side of the patient's face.

81. The positioning and stabilising structure according to claim 74 further comprising at least one headgear strap attached to the conduit.

82. A positioning and stabilising structure configured to hold a seal-forming structure of a patient interface in a sealing position on a patient's face, the positioning and stabilising structure comprising:
a conduit configured to permit delivery of a flow of air at a therapeutic pressure into a plenum chamber of the patient interface through an inlet formed in the plenum chamber, wherein the conduit comprises a patient facing side and a non-patient facing side; and
a pad comprising a conduit facing surface, an opposing patient facing surface, opposing lateral edges and opposing end edges, wherein the conduit facing surface of the pad is attached to an outer surface of the conduit on the patient facing side such that the patient facing surface of the pad contacts the patient's head and/or face in use, and
wherein the outer surface of the conduit comprises a first outer surface region and a second outer surface region, wherein the outer surface of the conduit is configured such that the first outer surface region is recessed from the second outer surface region, wherein the pad is positioned to lie over the second outer surface region, and wherein the outer surface of the conduit is configured such that at least one of the edges of the pad is positioned, in use, to face in a direction extending away from the patient's face and/or head.

83. The positioning and stabilising structure according to claim 82, wherein the conduit comprises a connecting wall which connects the first outer surface region and the second outer surface region.

84. The positioning and stabilising structure according to claim 82, wherein the pad is positioned to lie over the second outer surface region such that at least one of the edges of the pad is positioned on the first outer surface region.

85. The positioning and stabilising structure according to claim 82, wherein the conduit comprises first and second connecting walls connecting lateral edges of the first outer surface region with respective lateral edges of the second outer surface region, and wherein the pad is positioned on the second outer surface region between the first and second connecting walls, wherein the pad is positioned to lie over the second outer surface region such that at least one of the edges of the pad is positioned on the connecting wall.

86. The positioning and stabilising structure according to claim 82, wherein the conduit comprises first and second conduits, wherein the positioning and stabilising structure is configured such that, in use, the first conduit is positioned on one side of the patient's face and the second conduit is positioned on the other side of the patient's face.

87. The positioning and stabilising structure according to claim 82 further comprising at least one headgear strap attached to the conduit.

88. A positioning and stabilising structure configured to hold a seal-forming structure of a patient interface in a sealing position on a patient's face, the positioning and stabilising structure comprising:
a conduit configured to permit delivery of a flow of air at a therapeutic pressure into a plenum chamber of the patient interface through an inlet formed in the plenum chamber, wherein the conduit comprises a patient facing side and a non-patient facing side; and
a pad comprising a conduit facing surface, an opposing patient facing surface, opposing lateral edges and opposing end edges, wherein the patient facing surface of the pad is attached to an outer surface of the conduit on the patient facing side such that the patient facing surface of the pad contacts the patient's head and/or face in use, and
wherein the outer surface of the conduit comprises a first outer surface region, a second outer surface region, and a connecting wall which connects the first outer surface region and the second outer surface region, wherein the outer surface of the conduit is configured such that the first outer surface region is recessed from the second outer surface region, wherein the pad is positioned on the first outer surface region, and wherein the connecting wall and the second outer surface region are shaped such that a joint region between the connecting wall and the second outer surface region is concave-shaped.

89. The positioning and stabilising structure according to claim 88, wherein the outer surface of the conduit is configured such that at least one of the edges of the pad is positioned, in use, to face in a direction extending away from the patient's face and/or head.

90. The positioning and stabilising structure according to claim 88, wherein at least a portion of the first outer surface region is substantially flat and at least a portion of the second outer surface region is substantially convex-shaped.

91. The positioning and stabilising structure according to claim 88, wherein the first outer surface region is located on the patient facing side and the second outer surface region is located on the non-patient facing side.

92. The positioning and stabilising structure according to claim 88, wherein the joint region between the connecting wall and the second outer surface region is on the patient facing side.

93. The positioning and stabilising structure according to claim 88, wherein at least one of the edges of the pad is positioned on the joint region.

94. A patient interface comprising:
a plenum chamber pressurisable to a therapeutic pressure of at least 6 cmH₂O above ambient air pressure, wherein the plenum chamber comprises an inlet configured to receive a flow of air at the therapeutic pressure for breathing by a patient;
a seal-forming structure configured to form a seal with a region of the patient's face surrounding an entrance to the patient's airways, wherein the seal-forming structure is configured to maintain said therapeutic pressure in the plenum chamber throughout the patient's respiratory cycle in use; and
the positioning and stabilising structure according to claim 65.
